(19) United States
(12) Patent Application Publication
(10) Pub. No.: US 2011/0136751 A1
(43) Pub. Date: Jun. 9, 2011

(54) Use of Polyphenols in the Treatment of Cancer

(75) Inventors: José Estrela, Valencia (ES); Miguel A. Asensi, Alicante (ES)

(73) Assignee: Green Molecular, Valencia (ES)

(21) Appl. No.: 12/899,308

(22) Filed: Oct. 6, 2010

Related U.S. Application Data
(60) Provisional application No. 61/249,188, filed on Oct. 6, 2009.

(51) Int. Cl. A61K 31/352; A61K 31/353; A61K 31/7048; A61K 31/513; A61K 31/519; A61K 31/4745; A61P 35/00

(52) U.S. Cl. 514/27; 514/456; 514/274; 514/249; 514/283

(57) ABSTRACT

The present invention relates to polyphenol compounds, compositions thereof, and methods for treating or preventing cancer in a subject, the methods comprising co-administering to a subject an effective amount of two or more polyphenol compounds or a polyphenol composition thereof.

### BACKGROUND OF THE INVENTION

1. Field of the Invention

The present invention relates to polyphenol compounds and their use in methods for treating or preventing cancer in a subject, the methods comprising administering to a subject an effective amount of the polyphenol compounds.

2. Background Art

Cancer is second only to cardiovascular disease as a cause of death in the United States. The American Cancer Society estimated that in 2002, there were 1.3 million new cases of cancer and 555,000 cancer-related deaths. There are currently over 10 million living Americans who have been diagnosed with cancer and the NIH estimates the direct medical costs of cancer as over $100 billion per year with an additional $100 billion in indirect costs due to lost productivity—the largest such costs of any major disease.

Modalities useful in the treatment of cancer include chemotherapy, radiation therapy, surgery and biological therapy (a broad category that includes gene-, protein- or cell-based treatments and immunotherapy).

Despite the availability to the clinician of a variety of anticancer agents, conventional cancer therapies have many drawbacks. For example, almost all anticancer agents are toxic, and chemotherapy can cause significant, and often dangerous, side effects, including severe nausea, bone marrow depression, liver, heart and kidney damage, and immunosuppression. Additionally, many tumor cells eventually develop multi-drug resistance after being exposed to one or more anticancer agents. As such, single-agent chemotherapy is curative in only a very limited number of cancers. Most chemotherapeutic drugs act as anti-proliferative agents, acting at different stages of the cell cycle. Since it is difficult to predict the pattern of sensitivity of a neoplastic cell population, or the current stage of the cell cycle that a cell happens to be in, it is common to use multi-drug regimens in the treatment of cancer, which are typically more effective, but also more toxic than single-drug chemotherapy regimens.

Colorectal cancer (CRC) is the third most common cancer and the fourth most frequent cause of cancer deaths worldwide (1). Treatment of patients with recurrent or advanced CRC depends on the location of the disease. For patients with locally recurrent and/or liver-only and/or lung-only metastatic disease, surgical resection, if feasible, is the only potentially curative treatment; whereas patients with unrespectable disease are treated with systemic chemotherapy (www.cancer.gov). Currently, several first-line and second-line chemotherapy regimens are available that can be used in patients with recurrent or advanced CRC. The newer CRC chemotherapy schemas are serving as the platform on which combined novel targeted agents are based. Exemplary accepted first-line regimens include irinotecan-based (IFL, FOLFIRI, AIO) and oxaliplatin-based (FOLFOX4, FOLFOX6) (www.cancer.gov). Combined chemotherapy and radiation therapy is used in rectal cancer-bearing patients, although improvements in the outcome of colon cancer-bearing patients treated with radiation therapy have not been proved (www.cancer.gov). Survival for patients with advanced CRC is approximately 2 years on average and there is an ongoing need for the identification of new therapeutic agents and/or treatment strategies (2).

NF-κB is widely used by eukaryotic cells as a regulator of genes that control cell proliferation and cell survival. NF-κB regulates anti-apoptotic genes especially TRAF1 and TRAF2 and thereby checks the activities of the caspase family of enzymes which are central to most apoptotic processes. Active NF-κB turns on the expression of genes that keep the cell proliferating and protect the cell from conditions that would otherwise cause it to die via apoptosis. Thus, defects in NF-κB result in increased susceptibility to apoptosis leading to increased cell death. Conversely, overexpression of NF-κB or constitutively active NF-κB promote cell survival. As such, many different types of human tumors have misregulated NF-κB: NF-κB is constitutively active.

In unstimulated cells, the NF-κB dimers are sequestered in the cytoplasm by a family of inhibitors, called IκBs (Inhibitor of KB), which are proteins that contain multiple copies of a sequence called ankyrin repeats. By virtue of their ankyrin repeat domains, the IκB proteins mask the nuclear localization signals (NLS) of NF-κB proteins and keep them sequestered in an inactive state in the cytoplasm.

Activation of the NF-κB is initiated by the signal-induced degradation of IκB proteins. This occurs primarily via activation of a kinase called the IκB kinase (IKK). IKK is composed of a heterodimer of the catalytic IKK alpha and IKK beta subunits and a “master” regulatory protein termed NEMO (NF-κB essential modulator) or IKK gamma. When activated by signals, usually coming from the outside of the cell, the IκB kinase phosphorylates two serine residues located in an IκB regulatory domain. When phosphorylated on these serines (e.g., serines 32 and 36 in human IκBα), the IκB inhibitor molecules are modified by a process called ubiquitination, which then leads them to be degraded by a cell structure called the proteasome.

In tumor cells, NF-κB is active either due to mutations in genes encoding the NF-κB transcription factors themselves or in genes that control NF-κB activity (such as IκB genes); in addition, some tumor cells secrete factors that cause NF-κB to become active. Blocking NF-κB can cause tumor cells to stop proliferating, to die, or to become more sensitive to the action of anti-tumor agents. Thus, NF-κB is the subject of much active research among pharmaceutical companies as a target for anti-cancer therapy.

Bcl-2 derives its name from B-cell lymphoma 2. It is one of 25 genes in the Bcl-2 family known to date. The Bcl-2 family of genes governs mitochondrial outer membrane permeabilization (MOMP) and can be either pro-apoptotic (Bax, BAD, Bak and Bok) or anti-apoptotic (including Bcl-2 proper, Bcl-xL, and Bcl-w). The Bcl-2 gene has been implicated in a number of cancers, including melanoma, a malignant tumor of melanocytes which are found predominantly in skin, the bowel and the eye (e.g., uveal melanoma), as well as breast, prostate, and lung carcinomas. The gene is also implicated in schizophrenia and autoimmunity. There is some evidence indicating that abnormal expression of Bcl-2 and increased expression of caspase-3 may lead to defective apoptosis, which can promote cancer cell survival. Thus, Bcl-2 is also thought to be involved in resistance to conventional cancer treatment.

Different polyphenolic compounds of natural origin, such as trans-resveratrol (trans-3,5,4′-trihydroxystilbene, t-RESV), have been studied for their potential antitumor properties (3). Cancer chemopreventive activity of t-RESV was first reported by Jang et al. (4). However, anticancer properties of t-RESV are limited due to its low systemic bioavailability (5). Thus, structural modifications of the t-RESV molecule appeared necessary in order to increase the bioavailability while preserving its biological activity. Resveratrol has also been produced by chemical synthesis[1] and is sold as a nutritional supplement derived primarily from Japanese knotweed.

Trans-pterostilbene (trans-3,5-dimethoxy-4′-hydroxy-trans-stilbene, t-PTER), TMS (3,4′,5-reimwrhoxzy-trans-stilbene), 3,4′,4-DH-5-MS (3,4′-dihydroxy5-methoxy-trans-stilbnene) and 3,5-DH-4′MS (3,5-dihydroxy-4′-,ethoxy-trans-stilbene) are compounds chemically related to resveratrol. Quercetin (3,3′,4′,5,6-pentahydroxyflavone, QUER) is a plant-derived flavonoid, and has been used as a nutritional supplement. Quercetin has been shown to have anti-inflammatory and antioxidant properties and is being investigated for a wide range of potential health benefits.

In an earlier study, t-PTER and QUER showed in vivo longer half-life than t-RESV, and that combination of the two compounds inhibited metastatic growth of the malignant murine B16 melanoma F10 (B16M-F10) (6). t-PTER and QUER inhibited bcl-2 expression in B16M-F10 cells. At the molecular level, natural polyphenols (PFs) have been reported to modulate a number of key elements in cellular signal transduction pathways linked to the apoptotic process (caspases and bcl-2 genes) (7). Moreover, recent reports showed that polyphenolic compounds from blueberries, tea, or red wine can inhibit human colon cancer cell proliferation and induce apoptosis in vitro (8-10). Nevertheless, whether natural PFs may have useful applications in oncotherapy, and in CRC therapy in particular, remains to be investigated.

Accordingly, there exists a need for the prevention and treatment of colon cancer and other types of cancer. This invention addresses that need.

The recitation of any reference in this application is not an admission that the reference is prior art to this application.

### BRIEF SUMMARY OF THE INVENTION

In one embodiment, the present invention is directed to a method for treating cancer in a subject comprising, co-administering to said subject a therapeutically effective amount of pterostilbene and quercetin, wherein said cancer is characterized by overexpression or constitutive activation of NF-κB or Bcl-2. The method of treatment can have a cytostatic and/or cytotoxic effect on the cancer cells. In one embodiment, further comprises administering an additional therapeutic agent. The additional therapeutic agent can be a polyphenol other than pterstilbene or quercetin. In one embodiment, the additional polyphenol is selected from the group consisting of: TMS, 3,4′,4-DH-5-MS, 3,5-DH-4′MS, catechin, caffeic, hydroxytyrosol, rutin, and quercitrin. In a specific embodiment, the additional polyphenol is resveratrol.

In one embodiment, the method of the invention is used to treat a cancer selected from the group consisting of skin cancer, colon cancer, advanced colorectal cancer, breast cancer, prostate cancer, lung cancer, uveal melanoma, brain cancer, lung cancer, bone cancer, pancreas cancer, fibrosarcoma and rhabdomyosarcoma. In a specific embodiment, the cancer is colon cancer or advanced colorectal cancer. In another embodiment, the method of the invention further comprises treating said subject with chemotherapy or a radiation therapy. In one embodiment, the treatment includes both chemotherapy and radiation therapy. In a particular embodiment, the treatment cases partial or total regression of the cancer. In another embodiment, the method of treatment of the invention causes no systemic toxicity in a subject. In one embodiment, the chemotherapy uses an agent selected from the group consisting of: oxaliplatin, fluorouracil, leucovorin, 5-fluorouracil, leucovorin, and irinotecan. In a particular embodiment, the chemotherapy is an irinotecan-based chemotherapy or an oxaliplatin-based chemotherapy. Another particular embodiment of chemotherapy comprises administering to said subject a combination of oxaliplatin, fluorouracil and leucovorin, or a combination of 5-fluorouracil, leucovorin, and irinotecan.

In one embodiment of the invention, the pterostilbene and quercetin are administered orally. In an alternative embodiment, the pterostilbene and quercetin are administered intravenously.

A particular embodiment of the invention is directed to a method for treating colorectal cancer in a subject comprising co-administering to said subject a therapeutically effective amount of pterostilbene and quercetin, wherein said treatment inhibits cancer cell growth or kills cancer cells. In one embodiment, the subject is also treated with a radiation therapy or a chemotherapy. In a particular embodiment, the subject is treated with both radiation therapy and chemotherapy. The chemotherapy can be an irinotecan-based chemotherapy or an oxaliplatin-based chemotherapy. In one embodiment, the chemotherapy comprises administering to said subject a combination of oxaliplatin, fluorouracil and leucovorin, or a combination of 5-fluorouracil, leucovorin, and irinotecan. In a preferred embodiment, the method of the invention causes partial or total regression of the cancer in said subject.

In a specific embodiment of the invention, the treatment delivers a dose of quercetin of 800 mg/m² and a dose of pterostilbene of 800 mg/m² to said subject. In one embodiment, the pterostilbene and quercetin can be administered concurrently or sequentially.

### BRIEF DESCRIPTION OF THE DRAWINGS/FIGURES

The patent or application file contains at least one drawing executed in color. Copies of this patent or patent application publication with color drawing(s) will be provided by the Office upon request and payment of the necessary fee.

FIG. 1 shows in vitro inhibition of HT-29 cell growth by t-PTER and QUER at bioavailable concentrations. HT-29 cells were cultured as described in Example 1. t-PTER (40 μM), and QUER (20 μM) were added once per day (at 24-h intervals), starting 23 h after seeding. PFs were added 5 times along the culture time and were present, after each addition, for only 60 min. After that 60-min period in the culture flasks were washed out (3 times with PBS) and the medium renewed (controls received identical treatment). Cell growth (A) and viability on day 6 after seeding (B) are shown. Results are means±SD of 6-7 different experiments in each experimental condition. *P<0.01, +P<0.05 comparing each value versus controls (where basal medium was added instead of PFs).

FIG. 2 shows the inhibition of HT-29 xenograft growth by t-PTER and QUER. Tumor growth was measured during a 30-day period. Tumor volume, one week after inoculation and before PF administration, was of 56+15 mm3. A. PFs were administered i.v. at a dose of 10-30 mg/kg of body weight (one injection per day, starting one week after tumor inoculation). B. Growth profile of control () and t-PTER- and QUER (∘) (20 mg of each PF/kg)-treated HT-29-bearing mice. Data are means±S.D. of 18-20 mice per group. The significant test refers, for all groups, to the comparison between PTER and/or QUER and controls (treated with physiological saline) (*P<0.01).

FIG. 3 shows the expression of pro-death and anti-death Bcl-2 genes and of oxidative stress-related enzyme genes. HT-29-GFP xenograft-bearing mice were treated with t-PTER and QUER (20 mg each/kg of body weight) as indicated in the caption to FIG. 2. Tumor cells were isolated by laser microdissection (as indicated under “Materials and Methods”) 20 days after tumor inoculation. The data, expressing fold change (see under “Materials and Methods” for calculations), show mean values±S.D. for 9-10 different experiments (*P<0.01 for all genes displayed comparing t-PTER- and QUER-treated HT-29-GFP-bearing mice versus physiological saline-treated controls). We found no significant differences in expression of Bcl-2 genes and oxidative stress-related enzyme genes when in vitro cultured control HT-29 and HT-29-GFP cells were compared (not shown).

FIG. 4 shows t-PTER and QUER inhibit NF-κB. HT-29 cells were cultured and treated with PFs as indicated in the caption to FIG. 1. (A) NF-κB binding to DNA (measured 3 h after the last PF addition). Recombinant human TNFα (0.5 nM, Sigma, St. Louis, Mo.) was added 6 h before the last PF addition. IκBα (B) and P-IκBα (C) analysis by western blot (lane 1, TNFα; lane 2, control; lane 3, t-PTER+QUER). Relative densitometric intensities for protein bands from western blots of IκBα and P-IκBα) were normalized to β-actin (black bar, TNFα; white bar, control; grey bar, t-PTER+QUER). Results are means±SD of 5 different experiments in each experimental condition. *P<0.01 comparing each value versus controls (where basal medium was added instead of PFs and/or TNFα).

FIG. 5 shows the effect of siRNA-induced NF-κB p65 depletion on NF-κB binding to DNA and bcl-2 expression. Transfection of siRNA was performed as explained under “Materials and Methods”. (A) western blot analysis of p65 in cells transfected with p65 siRNA, or NS siRNA. For comparison NF-κB binding to DNA (B) and bcl-2 expression (RT-PCR) (C) were determined in HT-29 cells transfected with p65 siRNA or treated with dehydroxymethylepoxyquinomicin (DHMEQ). For this purpose cultured HT-29 cells (24 h after seeding) were incubated in the absence or in the presence of 10 μg DHMEQ/ml, and NF-κB activation and bcl-2 expression were measured 2 h and 12 h, respectively, after removing the inhibitor. The data show mean values±S.D. for 4-5 different experiments (*P<0.01, comparing all values versus controls).

FIG. 6 shows the effect of siRNA-induced SP1 and AP2 depletion on the induction of SOD2 expression by t-PTER and QUER. Transfection of siRNA was performed as explained under “Materials and Methods”. HT-29 cells were cultured and treated with PFs as indicated in the caption to FIG. 1. Western blot analysis of SP1(A) and AP2 (B) in cells transfected with SP1 siRNA (A), AP2 siRNA (B), or NS siRNA, and treated with PFs. Western blots displayed in (A) and (B) were found similar either before or after treatment with PFs (not shown). SOD2 expression was analysed by RT-PCR (see under “Materials and Methods”) 6 h after the last addition of t-PTER and QUER. The data, expressing fold change, show mean values±S.D. for 5-6 different experiments (*P<0.01, comparing all data versus control values in the absence of t-PTER+QUER treatment).

FIG. 7 shows the effects of t-PTER+QUER on O²⁻. and H₂O₂ generation by growing HT-29 cells.

FIGS. 8A through 8D show the inhibitory effects of PTER and RES on cancer cells.

FIG. 9A and FIG. 9B show cell cycle arrest induced by PTER and RES.

FIG. 10A and FIG. 10B show ncrosis induction by PTER and RES.

FIG. 11 shows aspase-3 activity in the presence of increasing concentration of PTER.

### DETAILED DESCRIPTION OF THE INVENTION

The present invention relates to Polyphenol compositions thereof and methods for treating and preventing cancer in a subject, the methods comprising administering to a subject an effective amount of a Polyphenol composition thereof.

### DEFINITIONS

The terms used herein having following meaning:

The term “co-administer” or “co-administering” refers to administer two or more compounds, for example two or more polyphenol compounds, to a subject. Such two or more compounds can be administered concurrently or sequentially, they can be administered via the same administration route (e.g., intravenous) or via different administration routes (e.g., oral and intravenous); they can be administered in the same or separate compositions.

A “subject” is a mammal, e.g., a human, mouse, rat, guinea pig, dog, cat, horse, cow, pig, or non-human primate, such as a monkey, chimpanzee or baboon. In one embodiment, a monkey is a rhesus. In another embodiment, a subject is a human.

The term “Polyphenol composition” refers to a polyphenol composition comprising at least one polyphenol compound or pharmaceutically acceptable salt thereof. Illustrative polyphenol compounds include, but are not limited to, pterostilbene, resveratrol, TMS (3,4′,5-reimwrhoxzy-trans-stilbene), 3,4′,4-DH-5-MS (3,4′-dihydroxy5-methoxy-trans-stilbnene), 3,5-DH-4′MS (3,5-dihydroxy-4′-,ethoxy-trans-stilbene), a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof.

In one embodiment, a polyphenol composition of the invention comprises two or more poylphenol compounds, for example, pterostilbene and quercetin. In another embodiment, a polyphenol composition of the invention comprises pterostilbene, quercetin and resveratrol. In yet another embodiment of the invention, a polyphenol composition comprises two or more polyphenol compounds or pharmaceutically acceptable salts thereof, and a physiologically acceptable carrier or vehicle.

The term “additive” when used in connection with the polyphenol compounds of the invention, means that the overall therapeutic effect of a combination of: (a) two or more polyphenol compounds or (b) one or more polyphenol compounds and one or more other anticancer agents, when administered as combination therapy for the treatment of cancer, is equal to the sum of the therapeutic effects of these agents when each is adminstered alone as monotherapy.

The term “synergistic” when used in connection with the polyphenol compounds, of the invention means that the overall therapeutic effect of a combination of: (a) two or more polyphenol compounds or (b) one or more polyphenol compounds and one or more other anticancer agents, when administered as combination therapy for the treatment of cancer, is greater than the sum of the therapeutic effects of these agents when each is administered alone as monotherapy.

The phrase “pharmaceutically acceptable salt,” as used herein, is a salt formed from an acid and a basic nitrogen group of a polyphenol compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-OH-3-naphthoate)) salts. The term “pharmaceutically acceptable salt” also refers to a salt prepared from a polyphenol compound having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-hydroxy substituted lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a polyphenol compound.

The term “a polyphenol compound” used herein includes the compound and any pharmaceutically acceptable salt thereof. It also includes a hydrate of a hydrate of the polyphenol compound.

The following abbreviations are used herein and have the following meanings: CRC, colorectal cancer; PF, polyphenol; t-PTER, trans-3,5-dimethoxy-4′-hydroxystilbene; QUER, quercetin; t-RESV, trans-3,5,4′-trihydroxystilbene; B16M-F10, B16 melanoma F10; DMEM, Dulbecco's modified Eagle's medium; GFP, green fluorescent protein; HT-29-GFP, HT-29 clones expressing GFP; SOD1, cuprozinc-type superoxide dismutase; SOD2, mangano-type superoxide dismutase; SOD2-AS, SOD2 antisense oligodeoxynucleotides; NF-κB, nuclear factor kappa B; P-IκBα, phosphorylated IκBα; siRNA, small interfering RNA; SP1, specificity protein 1; AP2, activating protein 2; NS siRNA; non specific siRNA; ROS, reactive oxygen species; DHMEQ, dehydroxymethylepoxyquinomicin.

The term “first-line chemotherapy” refers to the treatment that is usually given first in treating a particular cancer. For example, irinotecan-based therapy (IFL, FOLFIRI, and AIO) or oxaliplatin-based therapy (FOLFOX4 and FOLFOX6) is considered as first-line chemotherapy for colorectal cancer.

### Polyphenol Compounds and Polyphenol Compositions

As stated above, the present invention encompasses methods for treating or preventing cancer in a subject, the methods comprising co-administering to the subject an effective amount of polyphenol compounds.

Illustrative polyphenol compounds useful in the polyphenol compositions and present methods for treating or preventing cancer, include, but are not limited to the following compounds and pharmaceutically acceptable salts thereof: pterostilbene, resveratrol, TMS (3,4′,5-reimwrhoxzy-trans-stilbene), 3,4′,4-DH-5-MS (3,4′-dihydroxy5-methoxy-trans-stilbnene), 3,5-DH-4′MS (3,5-dihydroxy-4′-,ethoxy-trans-stilbene), a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof. The chemical structures of pterostilbene, resveratrol, and quercetin are shown below:

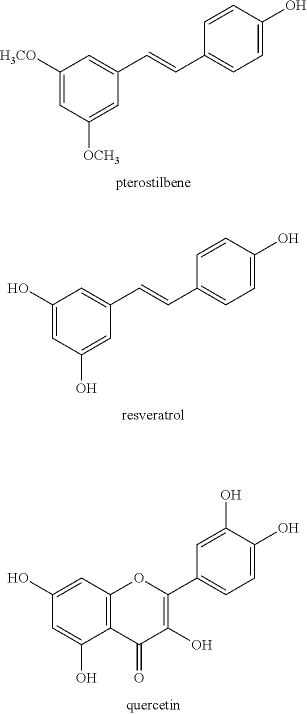

The polyphenol compounds may be purchased from commercial sources (e.g., Sigma Chemical, St. Louis, Mo.), prepared synthetically using methods well-known to one skilled in the art of synthetic organic chemistry, or extracted from natural sources using methods well-known to one skilled in the arts of chemistry and/or biology and/or related arts. For example, t-PTER can be synthesized following standard Wittig and Heck reactions (www.orgsyn.org), whereas QUER and resveratrol can be obtained from the Sigma Chemical Co. (St. Louis, Mo.). Alternatively, t-PTER can be purified from natural sources such as blueberries and grapes and QUER can be purified from capers, lovage, apples, tea, and red onion (e.g., 63, 64).

It is possible for some of the polyphenol compounds to have one or more chiral centers and as such these polyphenol compounds can exist in various stereoisomeric forms. Accordingly, the present invention is understood to encompass all possible stereoisomers.

It is possible for some of the polyphenol compounds to have geometric isomers, cis-(Z) and trans-(E). The present invention is understood to encompass all possible geometric isomers.

In one embodiment, a polyphenol compound is obtained from a natural product extract.

In one embodiment, a polyphenol composition comprises at least one polyphenol compound, in another embodiment, a polyphenol composition two or more polyphenol compounds. In another embodiment, the polyphenol composition further comprises a physiologically acceptable carrier or vehicle, and are useful for treating or preventing cancer in a subject.

In one embodiment, the polyphenol composition comprises pterostilbene and quercetin.

In another embodiment, the polyphenol composition comprises pterostilbene, quercetin and resveratrol.

In another embodiment, the polyphenol composition comprises pterostilbene, quercetin and a catechin.

### Treatment or Prevention of Cancer

The polyphenol compounds and compositions are useful for treating or preventing cancer.

In one embodiment, the invention provides a method for treating cancer in a subject, the method comprising co-administering to said subject a therapeutically effective amount of the polyphenol compounds pterostilbene and quercetin, or administering a composition comprising at least both these compounds In another embodiment, the method further comprises administering to the subject an additional polyphenol compound. In one embodiment, the additional polyphenol compound is resveratrol. In another embodiment, the additional polyphenol compound is selected from the group consisting of TMS (3,4′,5-reimwrhoxzy-trans-stilbene), 3,4′,4-DH-5-MS (3,4′-dihydroxy5-methoxy-trans-stilbnene), 3,5-DH-4′MS (3,5-dihydroxy-4′-,ethoxy-trans-stilbene), catechin, caffeic, hydroxytyrosol, rutin, and quercitrin. In one embodiment, said treatment inhibits cancer cell growth (i.e., the treatment is cytostatic), in another embodiment, the treatment kills cancer cells (i.e., is cytotoxic).

The polyphenol compounds can be administered concurrently or sequentially, they can be administered via the same administration route (e.g., intravenous) or via different administration routes (e.g., oral and intravenous); they can be administered in the same or separate compositions. In one embodiment, the method comprising administering a polyphenol composition comprising pterostilbene and quercetin. In another embodiment, the polyphenol composition comprises pterostilbene, quercetin and resveratrol.

It has been found that methods of treatment disclosed herein demonstrate cytostatic (i.e., inhibiting/blocking growth) and cytotoxic (i.e., killing) activities against tumor cells in vitro and in vivo. Thus, in one embodiment, the method for treating cancer inhibits cancer cell growth in the subject being treated. In another embodiment, the method prevents cancer progression in the subject being treated. In another embodiment, such treatment causes regression of such cancer in the subject being treated.

The polyphenol compositions disclosed herein are useful in treating solid tumors. In one embodiment, the polyphenol compounds are used in the treatment of a cancer selected from the group consisting of skin cancer, colon cancer, advanced colorectal cancer, breast cancer, prostate cancer, lung cancer, uveal melanoma, brain cancer, lung cancer, bone cancer, pancreas cancer, fibrosarcoma and rhabdomyosarcoma, or a combination thereof. In one embodiment, the polyphenol compounds are used for the treatment of breast cancer, colon cancer or advanced colorectal cancer.

As explained above, abnormal expression of Bcl-2 (e.g., overexpression) can lead to defective apoptosis, which can promote cancer cell survival. Thus, Bcl-2 is thought to be involved in resistance to conventional cancer treatment. It was demonstrated that the polyphenol treatments disclosed herein down-regulate bcl-2 expression or inhibit bcl-2 activity, for example, via inhibiting NF-kB activation. Thus, the polyphenol compounds and compositions disclosed herein are useful in treating cancer that is resistant to conventional therapy such as chemotherapy or radiation therapy.

Therefore, in one embodiment, the cancer being treated is characterized by overexpression or constitutive activation of NF-κB. In another embodiment, the cancer being treated is characterized by overexpression or constitutive activation of bcl-2.

In one embodiment, the cancer being treated or prevented is colon cancer or an advanced colorectal cancer.

In another embodiment, the cancer being treated or prevented is liver cancer.

In another embodiment, the cancer being treated or prevented is breast cancer. In another embodiment, the cancer being treated is prostate cancer.

Examples of cancers treatable or preventable using the polyphenol compounds and/or compositions include, but are not limited to, the cancers disclosed below and metastases thereof. Such cancer include solid tumors, including but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma liver cancer, bile duct carcinoma, choriocarcinoma, seminoma, embryonal carcinoma, Wilms'tumor, cervical cancer, uterine cancer, testicular cancer, small cell lung carcinoma, bladder carcinoma, lung cancer, epithelial carcinoma, skin cancer, melanoma, neuroblastoma, retinoblastoma, blood-borne cancers, including but not limited to: acute lymphoblastic leukemia (“ALL”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, acute and chronic leukemias: lymphoblastic, myelogenous, lymphocytic, myelocytic leukemias, Lymphomas: Hodgkin's disease, non-Hodgkin's Lymphoma, Multiple myeloma, Waldenstrom's macroglobulinemia, Heavy chain disease, Polycythemia vera, CNS and brain cancers: glioma pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor medulloblastoma.

In one embodiment the cancer is lung cancer, breast cancer, colorectal cancer, prostate cancer, a skin cancer, a brain cancer, a cancer of the central nervous system, ovarian cancer, uterine cancer, stomach cancer, pancreatic cancer, esophageal cancer, kidney cancer, liver cancer, a head and neck cancer, lung cancer, bone cancer, fibrosarcoma or rhabdomyosarcoma.

In one embodiment, the cancer is a solid tumor.

In a specific embodiment, the cancer is colorectal cancer.

In another specific embodiment the cancer is breast cancer.

In another specific embodiment the cancer is liver cancer.

In one embodiment, the subject has previously undergone or is presently undergoing treatment for cancer. Such previous treatments include, but are not limited to, prior chemotherapy, radiation therapy, surgery, or immunotherapy, such as a cancer vaccine.

### Combination Therapies for Cancer Treatment

In one embodiment, the present methods for coadministering two or more polyphenols to treat cancer or prevent cancer further comprise administering one or more other anticancer agents.

In one embodiment, the present invention provides a method for treating or preventing cancer in a subject, the method comprising coadministering (i) two or more polyphenol compounds or, alternatively, a composition comprising said two or more polyphenol compounds, and (ii) at least one other anticancer agent.

In one embodiment, the two or more polyphenol compounds or, alternatively, a composition comprising said two or more polyphenol compounds, and the at least one other anticancer agent are each administered in doses commonly employed when such agent is used alone for the treatment of cancer.

The dosing of two or more polyphenol compounds or a polyphenol composition comprising two or more polyphenol compounds, and (ii) another anticancer agent administered as well as the dosing schedule can depend on various parameters, including, but not limited to, the cancer being treated, the subject's general health, and the administering physician's discretion.

The polyphenol compounds or polyphenol compositions disclosed herein can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the at least one other anticancer agent to a subject in need thereof. In various embodiments, i) a polyphenol composition, and (ii) at least one anticancer agent are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart. In one embodiment, i) a polyphenol composition and (ii) at least one other anticancer agent are administered with 3 hours. In another embodiment, i) a polyphenol composition and (ii) at least one other anticancer agent are administered 1 minute to 24 hours apart.

In one embodiment, the method for treating cancer further comprises an effective amount of at least one other anticancer agent. This anticancer agent can be in the same or separate composition as that of the polyphenol compounds or the polyphenol compositions. In one embodiment, all the compounds are administered orally, in another embodiment, all the agents are administered intravenously. In one embodiment, the agents are administered via different routes. When the polyphenol compounds are comprised in a composition, the composition is useful for oral administration. In another embodiment, the composition is useful for intravenous administration.

Cancers that can be treated or prevented by administering and effective amount of (i) two or more polypehnol compounds or a polyphenol composition comprising two or more polypehnol compounds, and (ii) at least one other anticancer agent include, but are not limited to, the list of cancers set forth above.

In one embodiment the cancer is lung cancer, breast cancer, colorectal cancer, prostate cancer, a leukemia, a lymphoma, a non-Hodgkin's lymphoma, a skin cancer, a brain cancer, a cancer of the central nervous system, ovarian cancer, uterine cancer, stomach cancer, pancreatic cancer, esophageal cancer, kidney cancer, liver cancer, a head and neck cancer, lung cancer, bone cancer, fibrosarcoma or rhabdomyosarcoma.

In another embodiment, the cancer is colorectal cancer.

In still another embodiment the cancer is breast cancer.

In another embodiment the cancer is liver cancer.

The two or more polyphenol compounds or polyphenol compositions comprising the two or more polypehnol compounds, and the at least one other anticancer agents, can act additively or synergistically. A synergistic combination can allow the use of lower dosages of the polyphenol compounds and the at least one other anticancer agent, and/or less frequent dosages of the polyphenol compounds and the at least one other anticancer agents, and/or administering the polyphenol compounds and the at least one other anticancer agents less frequently. A synergistic effect can reduce any toxicity associated with the administration of the polyphenol compounds and the at least one other anticancer agents to a subject without reducing the efficacy in the treatment of cancer. In addition, a synergistic effect can result in the improved efficacy of these agents in the treatment of cancer and/or the reduction of any adverse or unwanted side effects associated with the use of either agent alone.

In one embodiment, the administration of an effective amount of two or more polyphenol compounds or a polyphenol composition comprising two or more polyphenol compounds, and another anticancer agent inhibits the resistance of a cancer to the other anticancer agent.

Suitable other anticancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the other anticancer agents useful in the methods and compositions of the present invention include, but are not limited to, a drug listed below or a pharmaceutically acceptable salt thereof. Alkylating agents Nitrogen mustards: Cyclophosphamide Ifosfamide Trofosfamide Chlorambucil Nitrosoureas: Carmustine (BCNU) Lomustine (CCNU) Alkylsulphonates: Busulfan Treosulfan Triazenes: Dacarbazine Procarbazine Temozolomide Platinum containing complexes: Cisplatin Carboplatin Aroplatin Oxaliplatin Plant Alkaloids Vinca alkaloids: Vincristine Vinblastine Vindesine Vinorelbine Taxoids: Paclitaxel Docetaxel DNA Topoisomerase Inhibitors Epipodophyllins Etoposide Teniposide Topotecan Irinotecan 9-aminocamptothecin Camptothecin Crisnatol Mitomycins: Mitomycin C Anti-metabolites Anti-folates: DHFR inhibitors: Methotrexate Trimetrexate IMP dehydrogenase Inhibitors: Mycophenolic acid Tiazofurin Ribavirin EICAR Ribonucleotide reductase Hydroxyurea Inhibitors: Deferoxamine Pyrimidine analogs: Uracil analogs: 5-Fluorouracil Fluoxuridine Doxifluridine Ralitrexed Cytosine analogs: Cytarabine (ara C) Cytosine arabinoside Fludarabine Gemcitabine Capecitabine Purine analogs: Mercaptopurine Thioguanine O-6-benzylguanine DNA Antimetabolites: 3-HP 2′-deoxy-5-fluorouridine 5-HP alpha-TGDR aphidicolin glycinate ara-C 5-aza-2′-deoxycytidine beta-TGDR cyclocytidine guanazole inosine glycodialdehyde macebecin II Pyrazoloimidazole Hormonal therapies: Receptor antagonists: Anti-estrogen: Tamoxifen Raloxifene Megestrol LHRH agonists: Goscrclin Leuprolide acetate Anti-androgens: Flutamide Bicalutamide Retinoids/Deltoids Cis-retinoic acid Vitamin A derivative: All-trans retinoic acid (ATRA-IV) Vitamin D3 analogs: EB 1089 CB 1093 KH 1060 Photodynamic therapies: Vertoporfin (BPD-MA) Phthalocyanine Photosensitizer Pc4 Demethoxy-hypocrellin A (2BA-2-DMHA) Cytokines: Interferon-.alpha. Interferon-.beta. Interferon-.gamma. Tumor necrosis factor Interleukin-2 Angiogenesis Inhibitors: Angiostatin (plasminogen fragment) antiangiogenic antithrombin III Angiozyme ABT-627 Bay 12-9566 Benefin Bevacizumab BMS-275291 cartilage-derived inhibitor (CDI) CAI CD59 complement fragment CEP-7055 Col 3 Combretastatin A-4 Endostatin (collagen XVIII fragment) Fibronectin fragment Gro-beta Halofuginone Heparinases Heparin hexasaccharide fragment HMV833 Human chorionic gonadotropin (hCG) IM-862 Interferon alpha/beta/gamma Interferon inducible protein (IP-10) Interleukin-12 Kringle (plasminogen fragment) Marimastat Metalloproteinase inhibitors (TIMPs) 2-Methoxyestradiol MMI 270 (CGS 27023A) MoAb IMC-1C11 Neovastat NM-3 Panzem PI-88 Placental ribonuclease inhibitor Plasminogen activator inhibitor Platelet factor-4 (PF4) Prinomastat Prolactin 16 kD fragment Proliferin-related protein (PRP) PTK 787/ZK 222594 Retinoids Solimastat Squalamine SS 3304 SU 5416 SU6668 SU11248 Tetrahydrocortisol-S Tetrathiomolybdate Thalidomide Thrombospondin-1 (TSP-1) TNP-470 Transforming growth factor-beta (TGF-b) Vasculostatin Vasostatin (calreticulin fragment) ZD6126 ZD 6474 farnesyl transferase inhibitors (FTI) Bisphosphonates Antimitotic agents: Allocolchicine Halichondrin B Colchicine colchicine derivative dolstatin 10 Maytansine Rhizoxin Thiocolchicine trityl cysteine Others: Protein Kinase G inhibitors: OSI 461 Exisulind Tyrosine Kinase inhibitors: Iressa Tarceva Dopaminergic neurotoxins: 1-methyl-4-phenylpyridinium ion Cell cycle inhibitors: Staurosporine Actinomycins: Actinomycin D Dactinomycin Bleomycins: Bleomycin A2 Bleomycin B2 Peplomycin Anthracyclines Daunorubicin Doxorubicin Idarubicin Epirubicin Pirarubicin Zorubicin Mitoxantrone MDR inhibitors: Verapamil Ca.sup.2+ATPase inhibitors: Thapsigargin

In one embodiment, the other anticancer agent is OSI 461.

In another embodiment, the other anticancer agent is Iressa.

In still another embodiment, the other anticancer agent is taxol.

In a further embodiment, the other anticancer agent is 5-fluorouracil.

In yet another embodiment, the other anticancer agent is a platinum-based anticancer agent.

In one embodiment, the platinum-based anticancer agent is cisplatin, carboplatin or oxaliplatin.

In one embodiment, two or more polyphenol compounds (or a polyphenol composition comprising two or more polyphenol compounds) are used in combination with first line cancer treatment regimens. Such first line treatment regimens include, but are not limited to irinotecan based (IFL, FOLFIRI, and AIO) and oxalipaltin-based regimen (FOLFOC4 and FOLFOX6). In one embodiment, two or more polyphenol compounds (or a polyphenol composition comprising two or more polyphenol compounds) are used in combination with an agent selected from the group consisting of oxaliplatin, fluorouracil, leucovorin, 5-fluorouracil, leucovorin, and irinotecan. In one embodiment, two or more polyphenol compounds (or a polyphenol composition comprising two or more polyphenol compounds) are administered in combination with oxaliplatin, fluorouracil and leucovorin. In one embodiment, two or more polyphenol compounds (or a polyphenol composition comprising two or more polyphenol compounds) is administered in combination with 5-fluorouracil, leucovorin, and irinotecan. In one embodiment, such combination therapy is used to treat a colon cancer, a colorectal cancer or an advanced colorectal cancer.

In one embodiment, two or more polyphenol compounds (or a polyphenol composition comprising two or more polyphenol compounds) can be administered to a subject that has undergone or is currently undergoing one or more additional anticancer therapies including, but not limited to, surgery, radiation therapy, or immunotherapy, such as cancer vaccines.

In one embodiment, the additional anticancer therapy is radiation therapy.

In another embodiment, the additional anticancer therapy is surgery.

In still another embodiment, the additional anticancer therapy is immunotherapy.

In a specific embodiment, the present methods for treating or preventing cancer comprise administering two or more polyphenol compounds (or a polyphenol composition comprising two or more polyphenol compounds) and a radiation therapy. The radiation therapy can be administered concurrently with, prior to, or subsequent to the polyphenol composition. In various embodiments, radiation therapy can be administered at least 30 minutes, one hour, five hours, 12 hours, one day, one week, one month, or several months (e.g., up to three months), prior or subsequent to administration of the polyphenol composition.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, in one embodiment the invention provides methods of treatment of cancer using two or more polyphenol compounds (or a polyphenol composition comprising two or more polyphenol compounds) in combination with chemotherapy and/or radiation therapy. In one embodiment, such combination therapies inhibit cancer cell growth and/or prevent cancer progression and/or result in regression of the cancer being treated. In one embodiment, such cancer includes colon cancer and advanced colorectal cancer. In one embodiment, such combination therapies do not cause systemic toxicity. In one embodiment, such systemic toxicity is measured by hematology and/or clinical chemistry standards. The subject being treated can, optionally, be treated with another anticancer therapy such as surgery, radiation therapy, or immunotherapy.

In one embodiment, the treatment comprises coadministering to a subject pterostilbene and quercetin to treat cancer in the subject. In another embodiment, the treatment further comprises administering an additional polyphenol compound. In one embodiment, the additional polyphenol compound is resveratrol. In another embodiment, the additional polyphenol compound is selected from the group consisting of TMS, 3,4′,4-DH-5-MS, 3,5-DH-4′MS, catechin, caffeic, hydroxytyrosol, rutin, and quercitrin.

In one embodiment, the polyphenol compounds are administered with a first-line chemotherapy regimen. Such first line regimen can be an irinotecan-based chemotherapy or an oxaliplatin-based chemotherapy. In one embodiment, the treatment comprises administering the polyphenol compounds in combination with oxaliplatin, fluorouracil and leucovorin. In another embodiment, the treatment comprises administering the polyphenol compounds in combination with 5-fluorouracil, leucovorin, and irinotecan. In one embodiment, the polyphenol compounds are administered in combination with a radiation therapy. In one embodiment, the polyphenol compounds are administered in combination with a chemotherapy and a radiation therapy. In one embodiment, the chemotherapy is a first-line chemotherapy. In one embodiment, the polyphenol compounds are pterostilbene and quercetin. In another embodiment, the polyphenol compounds are pterostilbene, quercetin and resveratrol. The polyphenol compounds, and/or chemotherapy, and/or radiation therapy can be administered concurrently or sequentially. The administration routes, dosages and/or frequency can be determined by a medical professional.

The present invention is also directed to the use of a combination of pterostilbene and quercetin for treatment of cancer in a subject, wherein said cancer is characterized by overexpression or constitutive activation of NF-κB or Bcl-2. Such a treatment can have a cytostatic and/or cytotoxic effect on the cancer cells. In one embodiment, the combination comprises an additional therapeutic agent, which can include a polyphenol other than pterostilbene or quercetin. In one embodiment, the polyphenol is selected from the group consisting of: TMS, 3,4′,4-DH-5-MS, 3,5-DH-4′MS, catechin, caffeic, hydroxytyrosol, rutin, and quercitrin. In another embodiment, the additional polyphenol is resveratrol.

The use can be for the treatment of a cancer selected from the group consisting of: skin cancer, colon cancer, advanced colorectal cancer, breast cancer, prostate cancer, lung cancer and uveal melanoma. In one embodiment, the cancer is colon cancer or advanced colorectal cancer.

The use can be in conjunction with chemotherapy or radiation therapy, or in conjunction with both. In one embodiment, the treatment causes partial or complete regression of a tumor. In one embodiment the use is in a treatment that has minimal or no systemic toxicity in a subject. In one embodiment, the chemotherapy is an irinotecan-based chemotherapy or an oxaliplatin-based chemotherapy. In another embodiment, the chemotherapy uses an agent selected from the group consisting of: oxaliplatin, fluorouracil, leucovorin, 5-fluorouracil, leucovorin, and irinotecan. In yet another embodiment, the chemotherapy comprises administering to said subject a combination of oxaliplatin, fluorouracil and leucovorin, or a combination of 5-fluorouracil, leucovorin, and irinotecan.

For the uses of the invention, the polyphenols, e.g., pterostilbene and quercetin, can be administered, for example, orally or intravenously. In one embodiment, the subject is a human. In another embodiment, the use of the invention is for a treatment that delivers a dose of quercetin of 800 mg/m² and a dose of pterostilbene of 800 mg/m² to said subject. The pterostilbene and quercetin can be administered concurrently or sequentially.

The present invention is also directed to use of a combination of pterostilbene and quercetin for making a medicament useful in the treatment of cancer in a subject, wherein said cancer is characterized by overexpression or constitutive activation of NF-κB or Bcl-2. Such a treatment can have a cytostatic and/or cytotoxic effect on the cancer cells. In one embodiment, the combination comprises an additional therapeutic agent, which can include a polyphenol other than pterostilbene or quercetin. In one embodiment, the polyphenol is selected from the group consisting of: TMS, 3,4′,4-DH-5-MS, 3,5-DH-4′MS, catechin, caffeic, hydroxytyrosol, rutin, and quercitrin. In another embodiment, the additional polyphenol is resveratrol.

The use can be for the treatment of a cancer selected from the group consisting of: skin cancer, colon cancer, advanced colorectal cancer, breast cancer, prostate cancer, lung cancer and uveal melanoma. In one embodiment, the cancer is colon cancer or advanced colorectal cancer.

The use can be in conjunction with chemotherapy or radiation therapy, or in conjunction with both. In one embodiment, the treatment causes partial or complete regression of a tumor. In one embodiment the use is in a treatment that has no systemic toxicity in a subject. In one embodiment, the chemotherapy is an irinotecan-based chemotherapy or an oxaliplatin-based chemotherapy. In another embodiment, the chemotherapy uses an agent selected from the group consisting of: oxaliplatin, fluorouracil, leucovorin, 5-fluorouracil, leucovorin, and irinotecan. In yet another embodiment, the chemotherapy comprises administering to said subject a combination of oxaliplatin, fluorouracil and leucovorin, or a combination of 5-fluorouracil, leucovorin, and irinotecan.

For the uses of the invention, the polyphenols, e.g., pterostilbene and quercetin, can be administered, for example, orally or intravenously. In one embodiment, the subject is a human. In another embodiment, the use of the invention is for a treatment that delivers a dose of quercetin of 800 mg/m² and a dose of pterostilbene of 800 mg/m² to said subject. The pterostilbene and quercetin can be administered concurrently or sequentially.

### Therapeutic/Prophylactic Administration and Compositions

Two or more polyphenol compounds (or a polyphenol composition comprising two or more polyphenol compounds) are advantageously useful in veterinary and human medicine. As described above, these polyphenol compounds and compositions are useful for treating or preventing cancer in a subject in need thereof.

The polyphenol compounds and compositions of the present invention can be in any form that allows for the compounds and compositions to be administered to a subject.

The polyphenol compounds can be formulated as polyphenol compositions for administration to a subject. A polyphenol composition can comprise at least one polyphenol compounds. For example, any of the above listed polyphenol compounds can be comprised in a same or separate polyphenol composition. In a particular embodiment, pterostilbene, quercetin can be comprised in a same polyphenol composition. In another particular embodiment, pterostilbene, quercetin and resveratrol can be comprised in the same polyphenol composition. In other embodiments, pterostilbene, quercetin and resveratrol can be comprised in separate polyphenol compositions.

When administered to a subject, a polyphenol composition can further comprise a physiologically acceptable carrier or vehicle. In one embodiment, the composition further comprises an additional anticancer agent. The present compositions can be administered orally. The compositions can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, dendrimers etc., and can be administered. Polyphenols or polyphenol compositions disclosed herein can also be associated with gold or platinum nanoparticles for targeting cancer cells (65).

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the polyphenol compound(s) contained in the polyphenol compositions into the bloodstream. The mode of administration is left to the discretion of the practitioner.

In one embodiment, the polyphenol compounds or compositions are administered orally, e.g., in an orally disintegrating tablet (ODT).

In another embodiment, the polyphenol compounds or compositions are administered intravenously.

In other embodiments, it can be desirable to administer the polyphenol compounds or compositions locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the polyphenol compounds or compositions into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant. In certain embodiments, the polyphenol compounds or compositions can be formulated as a suppository, with traditional binders and excipients such as triglycerides.

In another embodiment the polyphenol compounds or compositions can be delivered in a vesicle, in particular a liposome (see Langer, Science 249:1527-1533 (1990) and Liposomes in the Therapy of Infectious Disease and Cancer 317-327 and 353-365 (1989)).

In yet another embodiment the polyphenol compounds or compositions can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the polyphenol compounds or compositions, e.g., the spinal column, brain, heart, abdomen, thoracic cavity, skin, lung, or gastrointestinal tract, thus requiring only a fraction of the systemic dose.

The present compositions can optionally comprise a suitable amount of a physiologically acceptable excipient so as to provide the form for proper administration to the subject.

Such physiologically acceptable excipients can be liquids, such as water and oils, including those of petroleum, subject, vegetable, or synthetic origin, such as peanut oil, soybean oil, mineral oil, sesame oil and the like. The physiologically acceptable excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the composition is administered intravenously. Saline solutions and aqueous dextrose and glycerol solutions can also be employed as liquid excipients, particularly for injectable solutions. Suitable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsions, tablets, pills, pellets, capsules, capsules containing liquids, powders, sustained-release formulations, suppositories, aerosols, sprays, or any other form suitable for use. In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment the polyphenol compositions are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active platform driving a polyphenol composition are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the compositions can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the compositions are to be administered by infusion, they can be dispensed, for example, with an infusion bottle containing sterile pharmaceutical grade water or saline. Where the compositions are administered by injection, an ampule of sterile water for injection or saline can be provided so that the ingredients can be mixed prior to administration.

The compositions can be administered by controlled-release or sustained-release means or by delivery devices that are well known to one skilled in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active components using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to one skilled in the art, including those described herein, can be readily selected for use with the active components of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition of the invention comprises a minimal amount of one or more polyphenol compounds so as to treat or prevent cancer in a minimal amount of time. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the synergistic polyphenol compounds, and can thus reduce the occurrence of adverse side effects.

Controlled- or sustained-release compositions can initially release an amount of a polyphenol compound that promptly produces the desired therapeutic or prophylactic effect, and gradually and continually release other amounts of the polyphenol compounds to maintain this level of therapeutic or prophylactic effect over an extended period of time. To maintain a constant level of a polyphenol compound in the body, the polyphenol compound can be released from the dosage form at a rate that will replace the amount of polyphenol compound being metabolized and excreted from the body. Controlled- or sustained-release of a polyphenol compound or a polyphenol compound component of a polyphenol composition can be stimulated by various conditions, including but not limited to, changes in pH, changes in temperature, concentration or availability of enzymes, concentration or availability of water, or other physiological conditions or compounds.

The polyphenol compounds can administered to a subject at dosages from about 1 mg/m² to about 1000 mg/m², from about 100 mg/m² to about 700 mg/m², or from about 200 mg/m² to about 500 mg/m². The dosage administered is dependent upon various parameters, including, but not limited to, the cancer being treated, the subject's general health, and the administering physician's discretion. In specific embodiments, the total combined dosage of the dosage of each polyphenol compound administered to a subject is about 50 mg/m², about 75 mg/m², about 100 mg/m², about 125 mg/m², about 150 mg/m², about 175 mg/m², about 200 mg/m², about 225 mg/m², about 250 mg/m², about 275 mg/m², about 300 mg/m², about 325 mg/m², about 350 mg/m², about 375 mg/m², about 400 mg/m², about 425 mg/m², about 450 mg/m², about 475 mg/m², about 500 mg/m², about 525 mg/m², about 550 mg/m², about 575 mg/m², about 600 mg/m², about 625 mg/m², about 650 mg/m², about 675 mg/m², about 700 mg/m², about 725 mg/m², about 750 mg/m², about 775 mg/m², about 800 mg/m², about 825 mg/m², about 850 mg/m², about 875 mg/m², about 900 mg/m², about 925 mg/m², about 950 mg/m², about 975 mg/m², or about 1000 mg/m².

The amount of the polyphenol compounds that is effective in the treatment or prevention of cancer can be determined by standard clinical techniques. In addition, in vitro or in vivo assays can optionally be employed to help identify optimal dosage ranges. The precise dose to be employed will also depend on the identity of the synergistic polyphenol compounds being administered, route of administration, and the seriousness of the condition being treated and should be decided according to the judgment of the practitioner and each subject's circumstances in view of, e.g., published clinical studies. Suitable effective amounts for each synergistic polyphenol compound being administered, however, range from about 10 micrograms to about 5 grams. In certain embodiments, the effective amount is about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, and about 3.0 g. Dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months.

Suitable effective dosage amounts for the polyphenol compositions are based upon the total amount of the polyphenol compounds present in the compositions. For the polyphenol compositions disclosed herein, the total amount of polyphenol compounds can be within a range of from about 0.01 to about 100 w/w. The effective dosage amounts described herein refer to the total amounts of all polyphenol compounds administered. If one or more polyphenol composition is administered, the effective dosage amounts correspond to the combined amount of all polyphenol compounds in each of the polyphenol compositions administered.

In one embodiment, clinical applications may be derived from the studies disclosed in the examples since chemotherapy and radiotherapy doses used are within clinical standards, and since i.v. administration of t-PTER and QUER, at the doses herein reported, appears safe. The US FDA and the NCl have indicated that extrapolation of animal doses to human doses can be correctly performed through normalization to body surface area (60). Thus the human dose equivalent (HED) can be calculated by the following formula: HED (mg/kg)=animal dose (mg/kg)×(animal Km/human Km), using Km factors of 3 and 37 for mice and humans, respectively. For example, 20 mg QUER/kg in mice would be equivalent to 1.62 mg QUER/kg in humans. Given the structural similarities between these two PFs, it is reasonable to expect that the same principles and facts described above for QUER also apply for t-PTER

In one embodiment, the polyphenol compounds are administered concurrently to a subject in separate compositions. The polyphenol compounds may be administered to a subject by the same or different routes of administration.

In one embodiment, the polyphenol compounds and compositions disclosed herein can be administered in combination with conventional chemotherapy regimens. In one embodiment, the polyphenol compounds or compositions are used in combination with an agent selected from the group consisting of oxaliplatin, fluorouracil, leucovorin, 5-fluorouracil, leucovorin, and irinotecan. In another embodiment, a polyphenol composition is administered in combination with oxaliplatin, fluorouracil and leucovorin. In one embodiment, a polyphenol composition is administered in combination with of 5-fluorouracil, leucovorin, and irinotecan. In one embodiment, such combination therapy is used to treat a colon cancer, a colorectal cancer or an advanced colorectal cancer.

In one embodiment, the polyphenol compounds and compositions disclosed herein can be administered in combination with a radiation therapy. In one embodiment, the polyphenol compounds and compositions disclosed herein can be administered in combination with a chemotherapy and a radiation therapy.

When the polyphenol compounds or the polyphenol compounds and other chemotherapy agent(s) or the polyphenol compounds and radiation therapy are administered to a subject concurrently, the term “concurrently” is not limited to the administration of the polyphenol compounds at exactly the same time, but rather means that they can be administered to a subject in a sequence at the same time or within a time interval. When the polyphenol compounds or the polyphenol compounds and other chemotherapy agent(s) are not administered in the same composition, it is understood that they can be administered in any order to a subject in need thereof.

The present methods for treating or preventing cancer in a subject can further comprise administering another therapeutic agent to the subject being administered a polyphenol compound or polyphenol composition. In one embodiment the other therapeutic agent is administered in an effective amount.

Effective amounts of the other therapeutic agents are well known to one skilled in the art. However, it is well within the skilled artisan's purview to determine the other therapeutic agent's optimal effective amount range.

In one embodiment, the other therapeutic agent is an antiemetic agent. In another embodiment, the other therapeutic agent is a hematopoietic colony-stimulating factor.

In another embodiment, the other therapeutic agent is an agent useful for reducing any potential side effect of a synergistic polyphenol composition, a synergistic polyphenol compound, or another anticancer agent.

In another embodiment, the polyphenol compounds or polyphenol compositions can be administered prior to, at the same time as, or after an antiemetic agent, or on the same day, or within 1 hour, 2 hours, 12 hours, 24 hours, 48 hours or 72 hours of each other.

In another embodiment, the polyphenol compounds or polyphenol compositions can be administered prior to, at the same time as, or after a hematopoietic colony-stimulating factor, or on the same day, or within 1 hour, 2 hours, 12 hours, 24 hours, 48 hours, 72 hours, 1 week, 2 weeks, 3 weeks or 4 weeks of each other.

### Kits

The invention encompasses kits that can simplify the administration of a polyphenol compounds or composition(s) to a subject.

In one embodiment, the kit comprises a container containing an effective amount of the polyphenol compounds or polyphenol composition and an effective amount of 5-fluorouracil, leucovorin, and irinotecan. In another embodiment, the kit comprises a container containing an effective amount of the polyphenol compounds or a polyphenol composition and an effective amount of oxaliplatin, fluorouracil and leucovorin.

Kits of the invention can further comprise a device that is useful for administering the unit dosage forms. Examples of such a device include, but are not limited to, a syringe, a drip bag, a patch, an inhaler, and an enema bag.

The following examples are set forth to assist in understanding the invention and should not, of course, be construed as specifically limiting the invention described and claimed herein. Such variations of the invention, including the substitution of all equivalents now known or later developed, which would be within the purview of one skilled in the art, and changes in formulation or minor changes in experimental design, are to be considered to fall within the scope of the invention incorporated herein.

### EXAMPLES

### Example 1

### Materials and Methods

### Cell Culture

HT-29 human colon cancer cell lines were obtained from the American Type Culture Collection. HT-29 cells were grown in Dulbecco's modified Eagle's medium (DMEM) (Invitrogen, San Diego, Calif.), pH 7.4, supplemented with 10% fetal calf serum (Biochrom KG, Berlin, Germany), 100 units/ml penicillin and 100 μg/ml streptomycin. Cultures were maintained at 37° C. in a humidified atmosphere with 5% CO2. Cells were harvested by incubation for 5 min with 0.05% (w/v) trypsin (Sigma, St. Louis, Mo.) in PBS (10 mM sodium phosphate, 4 mM KCl, 137 mM NaCl), pH 7.4, containing 0.3 mM EDTA, followed by the addition of 10% calf serum to inactivate the trypsin. Cell numbers were determined using a Coulter Counter (Coulter Electronic, Inc., Miami, Fla.). Cellular viability was assessed, as previously reported (5), by measuring trypan blue exclusion and leakage of lactate dehydrogenase activity.

### Assessment of Cell Cycle Distribution

Analysis were performed using a MoFlo High-Performance Cell Sorter (DAKO, Copenhagen, Denmark). Fluorochrome excitation was performed with an argon laser tuned at 488 nm. Forward-angle and right-angle light scattering were measured. Data were acquired for 104 individual cells. Cell cycle phases were determined using the fluorescent DNA dye propidium iodide (final concentration, 5 μg/mL) (Molecular Probes, Leiden, The Netherlands) at 630 nm fluorescence emission (11).

### Cell Death Analysis

Apoptotic and necrotic cell death were distinguished by using fluorescence microscopy (12). For this purpose, isolated cells were incubated with Hoescht 33342 (Molecular Probes) (10 μM; which stains all nuclei) and propidium iodide (10 μM; which stains nuclei of cells with a disrupted plasma membrane), for 3 min, and analyzed using a Diaphot 300 fluorescence microscope (Nikon, Tokyo, Japan) with excitation at 360 nm. Nuclei of viable, necrotic, and apoptotic cells were observed as blue round nuclei, pink round nuclei, and fragmented blue or pink nuclei, respectively. About 1,000 cells were counted each time. DNA strand breaks in apoptotic cells were assayed by using a direct TUNEL labelling assay (Boehringer, Mannheim, Germany) and fluorescence microscopy following the manufacturer's methodology.

### Transfection of Green Fluorescent Protein

Long-term, stable expression of green fluorescent protein (GFP) in HT-29 cells was based on a previously described methodology (13). Briefly, 24 h before transfection, HT-29 cells were seeded in a 6-well tissue culture plate at a density of 5×105 in 2 ml of growth medium and incubated overnight. On the day of transfection, plasmid DNA (geneticine-resistant pEGFP-C1; Clontech, Mountain View, Calif.) was diluted into OPTIMEM (Invitrogen) and mixed with Lipofectamine 2000 (Invitrogen) according to supplier's protocol. Prior to transfection, the growth medium was replaced with 2 ml of OPTIMEM. DNA-lipofectamine complexes were added to the cells and incubated for 6 h. The transfection medium was then replaced by growth medium and cells were incubated for an additional 18 h-period. High-Performance Cell Sorting (DAKO) was used to select geneticine-resistant HT-29 clones expressing the GFP (HT-29-GFP) and showing high fluorescence emission.

### Tumor Xenografts

For HT-29 cancer cell xenograft experiments, female nu/nu nude mice (ages 6 to 8 weeks; Charles Rivers Laboratories, Wilmington, Mass.) were inoculated s.c. with 5×106 HT-29 or HT-29-GFP cells per mouse. Tumor volume was calculated based on two dimensions, measured using calipers, and was expressed in cubic millimeters according to the formula: V=0.5a×b2, where a and b are the long and the short diameters of the tumor, respectively. For histological analysis the surgical and xenograft tissue samples were fixed in 4% formaldehyde, paraffin embedded, and stained with hematoxilin & eosin and safran. Mice were monitored for at least 30 days after inoculation, and tumor measurements were taken on day 5, 10, 15, 20, 25, and 30. This study was conducted in compliance with international laws and policies (EEC Directive 86/609, OJ L 358. 1, Dec. 12, 1987; and NIH Guide for the Care and Use of Laboratory Animals, NIH Publ. No. 85-23, 1985).

### Laser Microdissection

Excised HT-29-GFP tumor samples were embedded in freezing medium ΔCT (Tissue-Tek, Electron Microscopy Sciences, Hatfield, Pa.) and immediately flash-frozen using isopentane and following Leica Microsystems' (Wetzlar, Germany) instructions to preserve RNA. Five-μm tissue slices were obtained using a Leica 2800E Frigocut Cryostat Microtome. Tumor cells were separated using a Leica LMD6000 Laser Microdissection System equipped with an automated fluorescence module.

RT-PCR and Detection of mRNA Expression

Total RNA was isolated using the trizol kit from Invitrogen and following manufacturer's instructions. cDNA was obtained using a random hexamer primer and a MultiScribe Reverse Transcriptase kit as described by the manufacturer (TaqMan RT Reagents, Applied Biosystems, Foster City, Calif.). A PCR master mix and AmpliTaq Gold DNA polymerase (Applied Biosystems) were then added containing the specific primers (Sigma-Genosys):

bax
F-CCAGCTGCCTTGGACTGT,
R-ACCCCCTCAAGACCACTCTT);
bak
(F-TGAAAAATGGCTTCGGGGCAAGGC,
R-TCATGATTTGAAGAATCTTCGTACC);
bad
(F-AGGGCTGACCCAGATTCC,
R-GTGACGCAACGGTTAAACCT);
bid
(F-GCTTCCAGTGTAGACGGAGC,
R-GTGCAGATTCATGTGTGGATG);
bik
(F-ATTTCATGAGGTGCCTGGAG,
R-GGCTTCCAATCAAGCTTCTG);
bim
(F-GCCCCTACCTCCCTACAGAC,
R-CAGGTTCCTCCTGAGACTGC);
bcl-2
(F-CTCGTCGCTACCGTCGTGACTTCG,
R-CAGATGCCGGTTCAGGTACTCAGTC);
bcl-w
(F-GGTGGCAGACTTTGTAGGTT,
R-GTGGTTCCATCTCCTTGTTG);
bcl-xl
(F-GTAAACTGGGGTCGCATTGT,
R-TGGATCCAAGGCTCTAGGTG);
Superoxide dismutase 1 (SOD1)
(F-TGAAGGTGTGGGGAAGCATTA,
R-TTACACCACAAGCCAAACGAC);
Superoxide dismutase 2 (SOD2)
(F-GGTAGCACCAGCACTAGCAGC,
R-GTACTTCTCCTCGGTGACGTTC);
Catalase (CAT)
(F-CCAGAAGAAAGCGGTCAAGA,
R-AACCTTCATTTTCCCCTGGG);
Glutathione peroxidase (GPx)
(F-CCTGGTGGTGCTCGGCTTCC,
R-CAATGGTCTGGAAGCGGCGG);
Glutathione reductase (GR)
(F-GTGCCAGCTTAGGAATAACCAG,
R-GTGAGTCCCACTGTCCCAATAG);
Thioredoxin reductase-1 (TrxR-1)
(F-CTCAGAGTAGTAGCTCAGTCC,
R-CATAGTCACACTTGACAGTGG);
glyceraldehyde-3P-dehydrogenase (GAPDH)
(F-CCTGGAGAAACCTGCCAAGTATG,
R-GGTCCTCAGTGTAGCCCAAGATG).

Real-time quantitation of the mRNA relative to GAPDH was performed with a SYBR Green I assay, and a iCycler detection system (Biorad, Hercules, Calif.). Target cDNA was amplified as follows: 10 min at 95° C., then 40 cycles of amplification (denaturation at 95° C. for 30 sec and annealing and extension at 60° C. for 1 min per cycle). The increase in fluorescence was measured in real time during the extension step. The threshold cycle (CT) was determined, and then the relative gene expression was expressed as: fold change=2^{-Δ(ΔCT)}, where ΔCT=CT target−CT GAPDH, and Δ (ΔCT)=ΔCT treated−ΔCT control.

### Ionizing Radiation

X rays were administered using a 6 KeV SL75 linear accelerator from Philips. For this purpose each mouse was anesthetized with nembutal (50 mg/kg i.p.), and fixed on a Perspex platform. Single fraction radiotherapy was administered at a rate of 2.0 Gy/min and the radiation beam was focused only on the tumor. The irradiated area was fixed to a maximum of 1.2 cm2, and the rest of the mouse had lead protection.

### Enzyme Assays

Tumor tissue was homogenized in 0.1M phosphate buffer (pH 7.2) at 4° C.

Superoxide dismutase (SOD) activity was measured as described by Flohé and Otting (14), using 2 mM cyanide in the assay medium to distinguish mangano-type enzyme (SOD2) from the cuprozinc type (SOD1).

### Antisense Oligodeoxynucleotides

Fully phosphorothioated 21-mer human SOD2 antisense oligodeoxynucleotide (SOD2-AS) was obtained from Sigma-Genosys (sequence: 5′-GGAACCUCACAUCAACGCGCA-3′). As a control, an equivalent but reversed phosphorothioated 21-mer sequence was purchased from the same source.

SOD2-AS were loaded onto the lipid surface of cationic gas-filled microbubbles by ion charge binding, as previously described (15). In vivo, uptake of a digoxigenin-labelled SOD2-AS was found in HT-29 tumour xenografts in nude mice following intratumoral injection of loaded microbubbles and subsequent exposure of the tumour to ultrasound (15).

Inhibition of SOD2 expression was verified by measuring the SOD2 activity and Western blot analysis. Tissue extracts were made by homogenization in a buffer containing 150 mM NaCl, 1 mM EDTA, 10 mM Tris-HCl, 1 mM phenylmethylsulfonyl fluoride, 1 μg/ml leupeptin, 1 μg/ml aprotinin, and 1 μg/ml pepstatin, pH 7.4. Fifty μg of protein [as determined by the Bradford assay (16)] were boiled with Laemmli buffer and resolved in 12.5% SDS-polyacrylamide gel electrophoresis. Proteins were transferred to a nitrocellulose membrane, and subjected to Western blotting with anti-human SOD2 monoclonal antibody (Sigma). Blots were developed using horseradish peroxidase-conjugated secondary antibody and enhanced chemiluminiscence (ECL system, Amersham, Arlington Heights, Ill.).

bcl-2 Gene Transfer and Analysis

The Tet-off gene expression system (Clontech, Palo Alto, Calif.) was used, as previously reported (17), to insert the human bcl-2 gene and for transfection into HT-29 cells following manufacturer's instructions. Bcl-2 protein was quantitated in the soluble cytosolic fraction by enzyme immunoassay (17) using a monoclonal antibody-based assay from Sigma (one unit of Bcl-2 was defined as the amount of Bcl-2 protein in 1000 non-transfected HT-29 cells).

### NF-κB DNA Binding

Evaluation of nuclear factor kappa B (NF-κB) p50/65 DNA binding activity in nuclear extracts of HT-29 cell samples was carried out to measure the degree of NF-κB activation. Nuclear extract were prepared as previously described (18). An enzyme linked immunosorbent assay (ELISA) was performed in line with the manufacturer's protocol for a commercial kit (Chemiluminiscent NF-κB p50/65 Transcription Factor Assay, Oxford Biomedical Research, Oxford, Mich.). Briefly, this chemiluminescence based sandwich type ELISA employs an oligonucleotide, containing the DNA binding NF-κB consensus sequence, bound to a 96-well ELISA plate. NF-κB present in the sample, binds specifically to the oligonucleotide coated on the plate. The DNA bound NF-κB is selectively recognized by the primary antibody, which, in turn, is detected by the secondary antibodyalkaline phosphatase conjugate. Antibodies anti-cyclin D1 were used as negative controls.

### Immunocytochemical Detection of NF-κB p65

HT-29 cells were grown in chamber slides and fixed with acetone. After two brief washes with PBS, slides were blocked with 5% normal goat serum for 1 h and then incubated with mouse monoclonal antibody anti-human p65 (Santa Cruz Biotechnology, Santa Cruz, Calif.). After overnight incubation, the slides were washed and then incubated with rabbit anti-mouse IgG-Alexa 594 (Molecular Probes) for 1 h and counterstained with Hoescht stain (50 ng/ml) for 5 min. Stained slides were analyzed using a TCS-SP2 confocal microscope (Leica Microsystems).

### Western Blot Analysis of I κBα and Phosphorylated-I κB-α

Whole cell extracts were made by freeze-thaw cycles in buffer containing 150 mM NaCl, 1 mM EDTA, 10 mM Tris-HCl, 1 mM phenylmethylsulfonyl fluoride, 1 μg/ml leupeptin, 1 μg/ml aprotinin, and 1 μg/ml pepstatin, pH 7.4. Fifty μg of protein [as determined by the Bradford assay (16)] were boiled in Laemmli buffer and resolved by 12.0% SDS-PAGE. Proteins were transferred to a nitrocellulose membrane and subjected to western blotting using mouse IgG1 monoclonal antibodies raised against human IκBα or a synthetic peptide containing phosphorylated serines at amino acid residues 32 and 36 of human P-IκB-α (Santa Cruz Biotechnology). Blots were developed using horseradish peroxidase-conjugated secondary antibody and enhanced chemiluminescence (ECL system).

### Transfection of Small Interfering RNA

HT-29 cells were seeded at a density of 106 cells per 9-cm dish. The first transfection was performed 12 h after seeding the cells. For each 9-cm dish, 50 Oligofectamine (Invitrogen) was added to 100 μl OPTIMEM (Invitrogen), and the solution was incubated at room temperature for 5-10 min. This was then added to a second solution or 800 μl OPTIMEM plus 50 μl 20 μM small interfering RNA (siRNA), and the mixture was incubated at room temperature for 15-20 min. Next, 4 ml OPTIMEM was added to the siRNA mixture to make a final volume of 5 ml, and this was added to the cells after rinsing them once with OPTIMEM. The transfection mixture was left for 4 h on the cells, after which 5 ml DMEM containing 20% FCS without antibiotics was added, and the cells were left in this mixture until they were trypsinized the following day. Two identical transfections were performed. The cells were trypsinized 24 h after the first transfection and were seeded into 9-cm dishes for the second transfection. After 48 h of incubation, following that second transfection, the cells were used for experiments. Human SP1 (specificity protein 1), AP2α (activating protein 2, alpha subunit), and p65 siRNAs, as well as a non specific (NS) siRNA which was used as a negative control, were obtained from Santa Cruz Biotechnology. In each case silencing was confirmed by immunoblotting.

### Western Blot Analysis of SP1, AP2, and p65

Human monoclonal antibodies anti-SP1, anti-AP2α, and anti-p65 were from Santa Cruz Biotechnology. Western blots were performed as described above.

### Fluorocytometric Analysis of Lymphocytes in Blood

Mononuclear cells were isolated from the blood by Ficoll-Hypaque (Pharmacia, Barcelona, Spain) centrifugation. Thereafter, 2×10⁵ freshly isolated leukocytes samples were suspended in 50 μl PBS containing 5% FCS and 0.1% Na-azide. Samples containing 5×10⁵ cells were incubated in PBS+5% FCS+0.1% Na-azide with rat anti-mouse CD3 (clone number KT3), rat anti-mouse CD4 (clone number YTS 191.1) and rat anti-mouse CD8 (clone number KT15) fluorescein labeled (Serotec, Oxford, UK) followed by streptavidin Cy5 coupled to R-phycoerytrin (PE) (Dako Cytomation), for 45 min on ice. Staining dot blot analysis was performed using a FACScan (Beckton Dickinson, Calif.). PE-conjugated anti-NK1.1 (cone number PK136) antibodies were used in double staining with anti-mouse CD3 labeled with FITC. Anti-mouse-kappa for detection of kappa-positive B cells was labeled with biotin (Southern Biotechnology Associates, Birmingham, Ala.) and detected with streptavidin-FITC (Jackson Immuno-Research, West Grove, Palo Alto, Calif.). Side scatter and forward scatter of dot plots were used to determine the gates of lymphocytes; PE- or FITC-labeled IgGs (Pharmingen, San Diego, Calif.) served as isotope controls for PE- or FITC-labeled antibodies. FACS analysis was done using FACSCalibur flow cytometer (Becton Dickinson, Erembodegem, Belgium). Data were analyzed using CELLQuest (Becton Dickinson).

### Measurement of O²⁻. and H₂O₂ Generation

O²⁻. generation was determined by flow cytometry using dihydroethidium (2 μg/ml; Molecular Probes, Leiden, The Netherlands). For this purpose cellular suspensions were diluted to 200,000 cell/ml. Analysis were performed with a MoFlo (DAKO) as previously described (19). Samples were acquired for 10,000 individual cells.

The assay of H₂O₂ production was based on the H₂O₂-dependent oxidation of the homovanillic acid (3-methoxy-4-hydroxyphenylacetic acid) to a highly fluorescent dimer (2,2′-dihydroxydiphenyl-5,5′-diacetic acid) that is mediated by horse-radish peroxidase (19).

### Statistical Significance

Data were analysed by Student's t test.

### Cell Inhibition Study

Cancer cells (0.2×10⁶ cells/well) were seeded in six well-plates and, 24 h later, were treated with PTER or RES (0-100 mM) (ethanol as solvent vehicle was at a conc. of 0.3%). Cell growth was analyzed using the Countess® Automated Cell Counter (Invitrogen). Results were expressed as relative proliferation index±SD (n=4) where control is 100.

### Cell Cycle Study

Cancer cells (0.2×10⁶ cells/well) were seeded in six well-plates and, 24 h later, were treated for 24 h with different concentrations (0-75 mM) of PTER or ReES. Cells were trypsinized and pelleted at 1000×g for 2 min. Cells were resuspended in PBS. Ethanol was added up to a final 70% conc. The DNA cellular content was analyzed by flow cytometry (10000 cell events were collected per sample). Results are expressed as % of total cells±SD (n=3).

### Necrosis Induction Measurement

Necrosis induction was evaluated by measuring lactate dehydrogenase (LDH) activity released to the extracellular medium. Cells were exposed to polyphenols for 24-72 h. Results are expressed as relative LDH activity±SD (n=3) where control is 100.

### Aspase-3 Activity Measurement

Aspase-3 activity was measured with Apo-ONE® Homogeneous Caspase-3 Assay (Promega). The assay was configured for 96 well plates (5000 cells/well). Cells were plated for 24 h. Capase-3/7 activities were evaluated 24 h after polyphenols addition. Fluorescence (a.u.) was expressed as relative index where control is 1. Results are expressed as relative proliferation index±SD (n=3).

### Example 2

### In Vitro Inhibition of HT-29 Growth by t-PTER and QUER

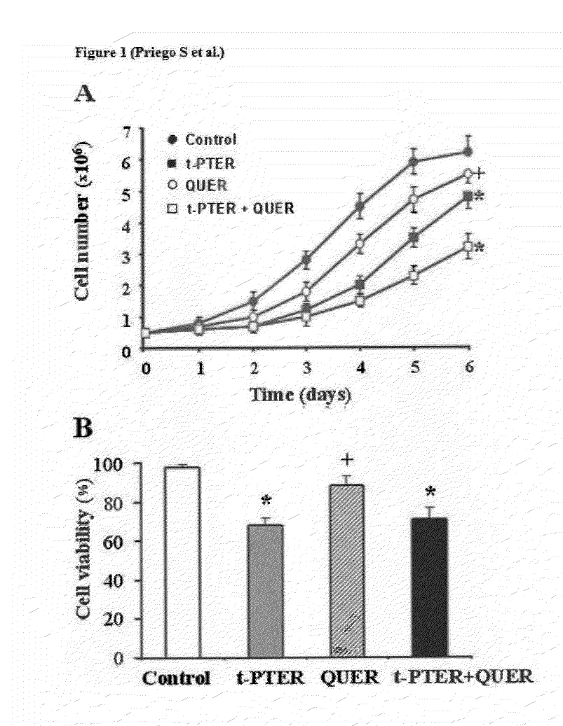

As shown in FIG. 1.A, when HT-29 human CRC cells were treated with PTER (40 μM) and QUER (20 μM) for a short period of time (60 min/day), the combination of t-PTER and QUER inhibited tumor growth in vitro (5 days after seeding) to ˜52% of control values. (˜47% and 35% of the cells accumulated in G2/M and S phases, respectively; whereas controls growing exponentially showed a cell cycle distribution of ˜50% in G0/G1, 26% in S, and 24% in G2/M) (n=7 in both cases, data not shown). Cell death analysis revealed that, 5 days after seeding, most non-viable cells (FIG. 1. B.) were apoptotic (>90% in all cases).

### Example 3

### PTER- and QUER-Induced Growth Inhibition of HT-29 Xenografts

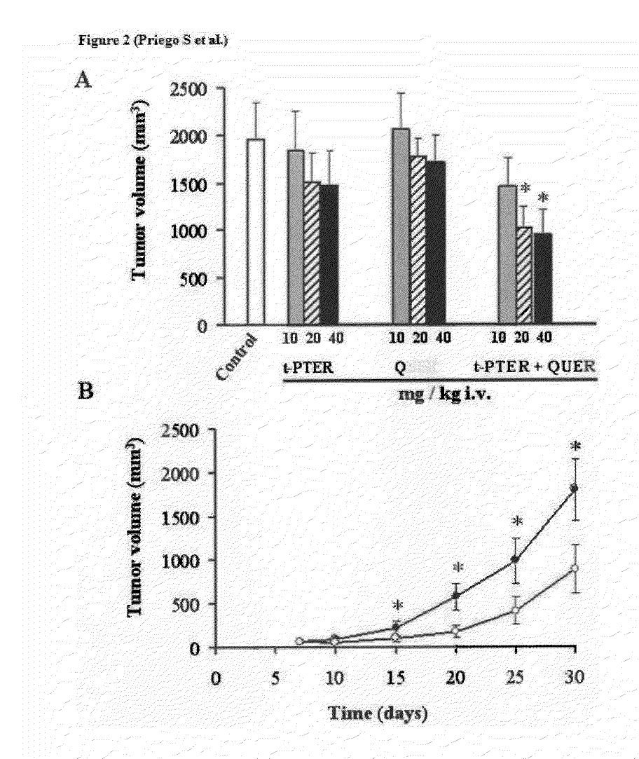

The effect of t-PTER and QUER on HT-29 CRC growth under in vivo conditions was investigated. As shown in FIG. 2, i.v. administration of t-PTER and QUER (20 mg of each PF/kg×day, administered every day at 10.00 a.m.) (dissolved as previously reported, see ref. (6)), inhibited CRC growth to ˜49% of control values (a % of inhibition that is coherent with the results reported in FIG. 1 under in vitro conditions). A lower dose (10 mg of each PF/kg×day) inhibited CRC growth to ˜87% of control values (n=10; P<0.05), whereas with a higher dose (30 mg of each PF/kg×day) the % of CRC growth inhibition, as compared to controls, was not significantly different to that found by administering 20 mg of each PF/kg×day (n=10 for each dose; not shown). Therefore in vivo administration of t-PTER and QUER, at clinically relevant doses, had a significant effect on human CRC growth.

t-PTER and QUER are pharmacologically safe since they have no organ-specific or systemic toxicity (including tissue histopathological examination and regular haematology and clinical chemistry data), even when administered i.v. at a high dose (e.g. 30 mg of each PF/kg×day×23 days) (Estrela et al., unpublished results).

### Example 4

### PFs and Chemoradiotherapy Eliminate HT-29 Tumors Growing In Vivo

We explored the effect of chemotherapy and radiotherapy in HT-29 tumor-bearing mice treated with t-PTER and QUER. FOLFIRI regimen (folic acid, 5-fluorouracil, irinotecan) and FOLFOX6 regimen (oxaliplatin, leucovorin, 5-fluorouracil) were selected as the best against HT-29 cells after in vitro drug screening (not shown). As shown in Table 1, PF administration improved the result of chemotherapy and/or radiotherapy on HT-29 xenograft growth. Tumor volume was smaller, in all conditions, when t-PTER and QUER were present in the treatment regimen (Table 1). The combination of t-PTER+QUER+X rays+the FOLFOX6 regimen was fully effective and achieved a complete regression of the tumor (Table 1). Mice survival was also studied for some of the conditions displayed in Table 1 and the results were as follows: 40±4 days for physiological saline-treated tumor-bearing mice, 52±5 (FOLFOX6), 59±4 (X rays+FOLFOX6), >120 days (in ˜85% of the mice treated with t-PTER+QUER+X rays+FOLFOX6) (n=20 mice in each case).

TABLE 1
Effect of natural PFs and chemoradiotherapy on HT-29 xenografts growth.

| | Tumor volume (mm³) | | | |
|---|---|---|---|---|
| | Physiological saline | | t-PTER + QUER | |
| X rays | − | + | − | + |
| Physiological saline | 1872 ± 344 | 1097 ± 201** | 1140 ± 263++ | 530 ± 178**++ |
| FOLFIRI | 643 ± 175 | 174 ± 60** | 316 ± 91++ | 77 ± 41**+ |
| FOLFOX6 | 410 ± 106 | 145 ± 69* | 70 ± 33++ | Nd |

Tumor volume one week after inoculation was, in all cases, of 50-70 mm3. t-PTER and QUER (20 mg each /kg × day × 23 days, starting one week after tumor inoculation) were administered i.v. Irinotecan (50 mg/kg) + leucovorin (120 mg/kg) + 5-fluorouracil (120 mg/kg) (FOLFIRI) or oxiplatin (30 mg/kg) + leucovorin (120 mg/kg) + 5-fluorouracil (120 mg/kg) (FOLFOX6) were administered i.v. on day 20; then 5-fluorouracil (180 mg/kg) was administered i.v. on days 25 and 28 after tumor inoculation. Animal doses of chemotherapy doses were calculated using NCI's human recommended doses for each drug (www.cancer.gov) and the conversion factor for mice published by the FDA (Center for Drug Evaluation and Research) (www.fda.gov). Mice received fractionated X ray therapy (5 Gy per day focused on the tumor - irradiation area was 1.0-1.2 cm2-) on days 22 and 24 after tumor inoculation [below maximum tolerated doses since the LD50 reported for mice subjected to whole body irradiations is of ~7.5-8 Gy (e.g. (62)]. Tumor volumes displayed in the table refer to those measured 30 days after inoculation. Data are means ± S.D. of 12-15 mice per group.
Non detectable: nd.
Histologic examination (see under xenografts in the “Materials and Methods” section) confirmed that, in 17 mice out of 20 (~85 %), the full treatment achieved a complete tumor regression. The significant test refers to the comparison between X ray treatment versus non-irradiated mice (*P < 0.05, **p < 0.01); and t-PTER + QUER administration versus treatments without PFs (+P < 0.05, ++P < 0.01).

### Example 5

### Evaluation of Therapy-Induced Systemic Toxicity

Complete blood cell count and standard blood chemistry were measured to evaluate the side effects of the treatment regimen that eliminated HT-29 xenografts from the majority of treated mice. As shown in Table 2, side effects included e.g. anemia, severe lymphopenia and neutropenia, and an increase of several tissue-damage—related enzyme activities in plasma, including aspartate aminotransferase, alanine aminotransferase, γ-glutamyl transpeptidase, alkaline phosphatase, and lactate dehydrogenase. However, in the mice cleared of tumor (85%, >120-day survival, see above), all hematologic and clinical chemistry measures practically returned to normal values measured in untreated, non-tumor-bearing mice by 30 days after treatment (Table 2).

TABLE 2
Hematology and clinical chemistry data in HT-29-bearing mice treated with
natural polyphenols and chemoradiotherapy.

| | | Tumor-bearing mice | | |
|---|---|---|---|---|
| | Non-tumor- | | | 30 days after |
| | bearing mice | +Vehicle control | +Full treatment | full treatment |
| Hematology | | | | |
| Hematocrit (%) | 37.5 ± 1.2 | 31.4 ± 2.7* | 22.3 ± 2.4*+ | 30.6 ± 2.0* |
| Hemoglobin (g/dl) | 12.8 ± 0.3 | 12.6 ± 0.5 | 8.0 ± 0.7*+ | 12.5 ± 0.4 |
| Erythrocites (10⁶/μl) | 8.4 ± 0.2 | 6.9 ± 0.3* | 4.6 ± 0.5*+ | 7.5 ± 0.2*+ |
| Platelets (10³/μl) | 470 ± 46 | 395 ± 37* | 123 ± 26*+ | 415 ± 35 |
| Leukocytes (10³/μl) | 2.4 ± 0.5 | 2.0 ± 0.3 | 0.4 ± 0.1*+ | 2.2 ± 0.3 |
| Neutrophils (10³/μl) | 1.0 ± 0.1 | 0.8 ± 0.2 | 0.1 ± 0.05*+ | 1.2 ± 0.2 |
| Lymphocytes (10³/μl) | 1.2 ± 0.2 | 1.1 ± 0.3 | 0.3 ± 0.1*+ | 0.9 ± 0.2 |
| % of CD3 | 1.4 ± 0.3 | 1.2 ± 0.2 | 1.2 ± 0.3 | 1.3 ± 0.3 |
| CD4 | 1.0 ± 0.1 | 1.0 ± 0.2 | 0.8 ± 0.2 | 0.9 ± 0.2 |
| CD8 | 0.5 ± 0.1 | 0.3 ± 0.1 | 0.3 ± 0.05 | 0.4 ± 0.1 |
| B cells | 54.9 ± 7.7 | 60.5 ± 6.4 | 66.7 ± 5.6 | 52.6 ± 7.6 |
| NK | 8.3 ± 2.0 | 2.5 ± 1.0* | 0.3 ± 0.1*+ | 7.2 ± 1.3+ |
| Monocytes (10³/μl) | 0.1 ± 0.05 | 0.05 ± 0.02 | 0.02 ± 0.005*+ | 0.1 ± 0.5 |
| Eosinophils (10³/μl) | 0.1 ± 0.05 | 0.05 ± 0.01 | 0.01 ± 0.002*+ | 0.05 ± 0.02 |
| Basophils (10³/μl) | 0.0 ± 0.0 | 0.0 ± 0.0 | 0.0 ± 0.0 | 0.0 ± 0.0 |
| Clinical chemistry | | | | |
| Urea (mg/dl) | 50.2 ± 3.0 | 51.3 ± 2.5 | 46.4 ± 3.9 | 55.8 ± 4.7 |
| Uric acid (mg/dl) | 2.0 ± 0.3 | 1.5 ± 0.2 | 0.5 ± 0.2*+ | 2.1 ± 0.4+ |
| Total protein (g/dl) | 4.2 ± 0.3 | 4.0 ± 0.2 | 3.9 ± 0.2 | 4.0 ± 0.3 |
| Albumin (g/dl) | 2.9 ± 0.2 | 2.6 ± 0.3 | 2.4 ± 0.3 | 2.7 ± 0.3 |
| Creatinin (mg/dl) | 0.5 ± 0.1 | 0.5 ± 0.04 | 0.6 ± 0.03 | 0.5 ± 0.1 |
| Glucose (mg/dl) | 160 ± 15 | 152 ± 26 | 116 ± 12* | 184 ± 31 |
| Total bilirubin (mg/dl) | 0.5 ± 0.2 | 0.4 ± 0.1 | 0.7 ± 0.1+ | 0.5 ± 0.1 |
| Direct bilirubin (mg/dl) | 0.1 ± 0.01 | 0.05 ± 0.02* | 0.2 ± 0.05*+ | 0.1 ± 0.05 |
| Aspartate aminotransferase (IU/l) | 178 ± 27 | 260 ± 39* | 517 ± 66*+ | 243 ± 31* |
| Alanine aminotransferase (IU/l) | 8.0 ± 3.2 | 52.4 ± 10.6* | 214 ± 47*+ | 36.5 ± 12.2* |
| γ-glutamyl transpeptidase (IU/l) | 2.1 ± 0.6 | 4.6 ± 2.0* | 20.5 ± 7.1*+ | 3.3 ± 1.2 |
| Alkaline phosphatase (IU/l) | 140 ± 20 | 153 ± 36 | 490 ± 77*+ | 167 ± 29 |
| Lactate dehydrogenase (IU/l) | 267 ± 40 | 424 ± 78* | 1066 ± 123*+ | 377 ± 45* |

Standard cell count and chemistry were measured in peripheral blood samples taken fron the saphena vein. Full treatment means the combination of t-PTER, QUER, FOLFOX6, and X rays (given as indicated in the caption of Table 1). Tumor-bearing mice were sacrified 1 or 30 days after finishing the full treatment, whereas controls treated with vehicle were sacrified 1 day after finishing the treatment. Data are means ± S.D. for 6-7 different mice in each experimental condition.
*P < 0.05 comparing tumor-bearing mice versus non-tumor-bearing mice;
+P < 0.05 comparing full treatment versus treatment with veh

### Example 6

### Gene Expression Profile of Bcl-2-Related Proteins and Antioxidant Enzymes in PF-Resistant HT-29 Cells

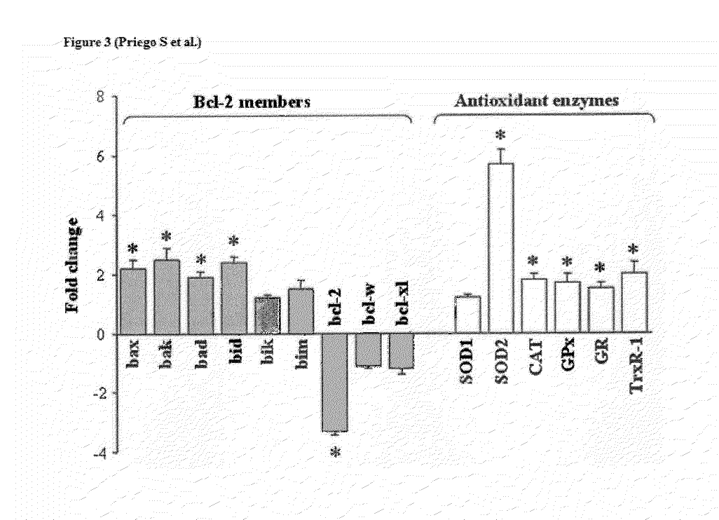

Natural PFs can regulate expression of apoptosis regulators (7). Bcl-2 family proteins are regulators of chemo- and radioresistance in cancer (21-23). Besides, enhanced antioxidant mechanisms in tumor cells have been implicated in chemoresistance, are radioprotectants, and lead to poor prognosis (24, 25). FIG. 3 shows tumor genes which are up- or down-regulated in the PF-treated HT-29-GFP-bearing mice compared with physiological saline-treated controls. The comparison revealed that treatment with the PF association promotes, preferentially, a decrease in bcl-2 (˜3.3-fold) and an increase in SOD2 expression (˜5.7-fold). (FIG. 3). SOD2 overexpression (FIG. 3) inhibits tumor cell proliferation (26); whereas the antiapoptotic Bcl-2 protein, which is down-regulated by PFs (FIG. 3), is among the molecules (including p53 mutants, Bcl-2, Neu3, and COX-2) which actively promote CRC cell survival (27). Therefore PF-induced inhibition of CRC growth associates with changes in expression of potential regulators of CRC growth and survival. Hence, it appears plausible that PF administration can modulate the effect of conventional therapy against CRC cells under in vivo conditions.

### Example 7

### SOD2 and Bcl-2 are Targets in the Mechanism Activated by the PF Association

Different mechanisms can influence CRC growth and/or survival (see above). Following the findings displayed in FIG. 3, SOD2 and Bcl-2 were selected to investigate their role in the increased drug and radiation anti-tumor efficacy found in combination with t-PTER and QUER. For this purpose we used two different strategies: intratumoral injection of a SOD2-AS to decrease the SOD2 activity in growing HT-29 cells; and genetic engineering to obtain bcl-2 overexpressing HT-29 cells (HT-29/Tet-bcl-2). As shown in Table 3.A, treatment of HT-29-bearing mice with t-PTER and QUER increased SOD2 activity (without affecting the SOD1). Besides, treatment of HT-29-bearing mice with the PF association decreased Bcl-2 levels (Table 3.A). Treatment with SOD2-AS decreased the SOD2 activity, but without affecting Bcl-2 levels (Table 3.A). In HT-29/Tet-bcl-2 cells, bcl-2 overexpression (as compared to HT-29 control cells), was the only difference (Table 3.A). Changes in SOD2 activity or Bcl-2 levels displayed in Table 3.A were confirmed by western blot analysis (not shown).

In vivo HT-29 and HT-29/Tet-bcl-2 cell growth was not significantly different (Table 3.B). However combined treatment with PFs and chemoradiotherapy was unable to induce a complete tumor regression in HT-29/Tet-bcl-2 xenografts or in HT-29 xenografts treated with the SOD2-AS (Table 3.B). These facts demonstrate that SOD2 up-regulation and Bcl-2 down-regulation facilitate the complete CRC regression reached by combination of PFs with chemoradiotherapy.

TABLE 3
Effect of SOD2 silencing and/or bcl-2 overexpression on HT-29 cell
resistance to treatment with natural PFs and chemoradiotherapy in vivo.
A

| | Units/mg protein | | | | | | | |
|---|---|---|---|---|---|---|---|---|
| | HT-29 | | | | HT-29/tet-Bcl-2 | | | |
| | −SOD-AS | | −SOD2-AS | | −SOD-AS | | +SOD2-AS | |
| | −PQ | +PQ | −PQ | +PQ | −PQ | +PQ | −PQ | +PQ |
| SOD2 | 1.35 ± 0.23 | 4.9 ± 0.56^{c} | 0.27 ± 0.056^{b} | 1.18 ± 0.24^{bc} | 1.12 ± 0.3 | 5.6 ± 0.64^{bc} | 0.33 ± 0.12 | 1.06 ± 0.25^{bc} |
| SOD1 | 6.24 ± 0.47 | 6.83 ± 0.83 | 5.77 ± 0.62 | 6.05 ± 0.75 | 5.49 ± 0.39 | 6.17 ± 0.77 | 6.3 ± 0.48 | 5.87 ± 0.55 |
| Bcl-2 | 21 ± 3 | 6 ± 2^{c} | 19 ± 3 | 7 ± 1^{c} | 75 ± 6^{a} | 53 ± 5^{ac} | 81 ± 9^{a} | 57 ± 6^{ac} |

B

Tumor volume (mm³)

| | HT-29 | HT-29/tet-Bcl-2 |
|---|---|---|
| Physiological saline | 1645 ± 317 | 1860 ± 412 |
| Chemoradiotherapy + PQ | nd^{b} | 217 ± 71^{ab} |
| SOD2-AS | 2337 ± 266^{b} | 2942 ± 387^{ab} |
| Chemoradiotherapy + PQ + SOD2-AS | 167 ± 46^{b} | 584 ± 129^{ab} |

Mice were inoculated cultured HT-29 or HT-29/Tet-bcl-2 cells.
PQ: t-PTER + QUER.
A. SOD2 and SOD1 activities and Bcl-2 levels in HT-29 and HT-29/Tet-bcl-2 xenograft samples obtained from control or from tumor-bearing mice treated with t-PTER + QUER (20 mg each/kg of body weight, as indicated in the caption to FIG. 2) and/or SOD2-AS (5 mg/kg of body weight each 3 days, starting 4 days after tumor inoculation). Intratumoral injection of SOD2-AS was performed as explained under “Materials and Methods”. A reversed-sequence control SOD2-AS was used for comparison, but results were not significantly different from those obtained in physiological saline-treated mice (not shown). Histopathological examination of the xenograft samples revealed that most tissue (>95% in all cases) corresponds to tumor cells.
Data are means ± S.D. of 8-10 mice per group.
^{a}P < 0.01 comparing HT-29/Tet-bcl-2 versus HT-29 tumors;
^{b}P < 0.01 comparing treatment with SOD2-AS versus treatment with physiological saline;
^{c}P < 0.01 comparing treatment with t-PTER + QUER (PQ) versus treatment with PS.
B. HT-29- or HT-29/Tet-bcl-2-bearing mice were treated with t-PTER + QUER, chemoradiotherapy (FOFOX6 and X rays) (as indicated in Table 1), and SOD2-AS (as indicated above). Tumor volume was measured 30 days after inoculation.
Non detectable: nd.
Data are means ± S.D. of 10-12 mice per group.
^{a}P < 0.01 comparing HT-29/Tet-bcl-2 versus HT-29 tumors;
^{b}P < 0.01 comparing all conditions versus physiological saline-treated controls.

### Example 8

### PFs down-Regulate bcl-2 Expression by Inhibiting NF-kB Activation

Nuclear factor kappa B (NF-κB) contributes to development and/or progression of malignancy by regulating the expression of genes involved in cell growth and proliferation, anti-apoptosis, angiogenesis, and metastasis (28). NF-κB may inhibit apoptosis in CRC cancer cells through activation of expression of anti-apoptotic genes, such as bcl-2 (29). In fact inactivation of NF-κB in different cancer cells has been demonstrated to blunt the ability of the cancer cells to grow (30).

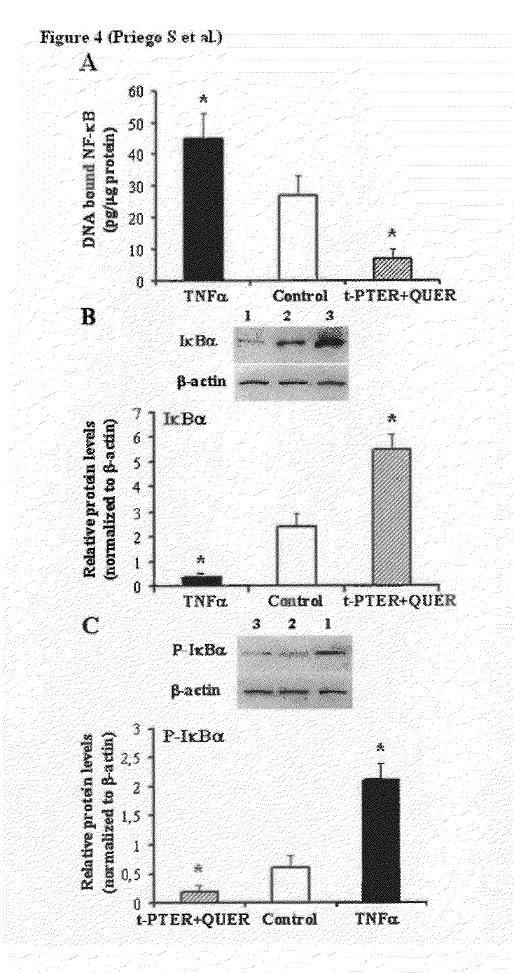

Some reports have suggested that natural PFs (e.g. the green tea constituent epigallocatechin 3-gallate) inhibit growth, in part, through blocking of the signal transduction pathways leading to activation of critical transduction factors such as NF-κB (31). Thus, we investigated if t-PTER- and QUER-induced down-regulation of bcl-2 was linked to the mechanism of NF-κB activation. As shown in FIG. 4.A, PFs decreased binding of NF-κB to the DNA as compared to controls [we found a linear correlation ([2>0.99) between relative light units and the amount of NF-κB]. Total cell extracts from HT-29 cells cultured in the presence of TNFα served as positive control (FIG. 4). Suppression of NF-κB activation was also confirmed by immunocytochemistry, since t-PTER and QUER inhibited nuclear translocation of p65 in HT-29 cells immunostained with antibody anti-p65 and then visualized with Alexa 594-conjugated second antibody (see under “Materials and Methods”).

Whether inhibition of NF-κB activation was due to inhibition of IκBα (the most prominent member of the IκB family in mammalian cells) degradation was examined next. As sown in FIG. 4, PFs also inhibited IκBα degradation (FIG. 4.B) and, in parallel, decreased the content of phosphorylated IκBα (FIG. 4.C)

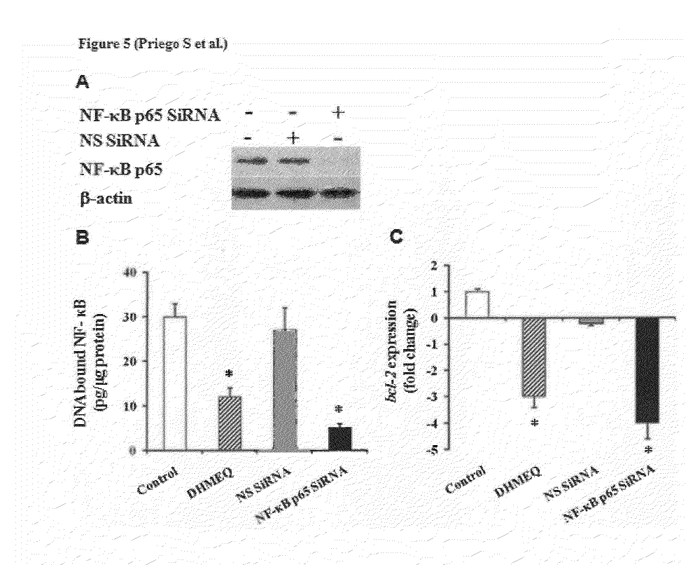

To investigate further if inhibition of NF-κB is fully or partially responsible of down-regulating bcl-2 expression, cultured HT-29 cells were treated with NF-κB p65-specific siRNA. Western blot analysis showed that p65 siRNA depletes the intracellular content of the protein (FIG. 5.A). Whereas, p65 siRNA or dehydroxymethylepoxyquinomicin [DHMEQ; which specifically inhibits nuclear translocation and activation of NF-κB (39)] induced a significant decrease in NF-κB binding to DNA (FIG. 5.B) and in bcl-2 (FIG. 5.C) expression which is similar to the decrease reported in FIG. 3 in HT-29 cells growing in mice treated with t-PTER and QUER. Therefore our results indicate that t-PTER- and QUER-induced down-regulation of bcl-2 is NF-κB dependent.

### Example 9

### PFs Up-Regulate SOD2 Expression Via a SP1-Dependent Mechanism

Transcriptional activation of human SOD2 mRNA, induced by t-PTER and QUER (FIG. 3), was examined to identify the responsive transcriptional regulator. Based on the results shown above, t-PTER- and QUER-induced up-regulation of SOD2 (FIG. 3) should involved an NF-κB-independent mechanism.

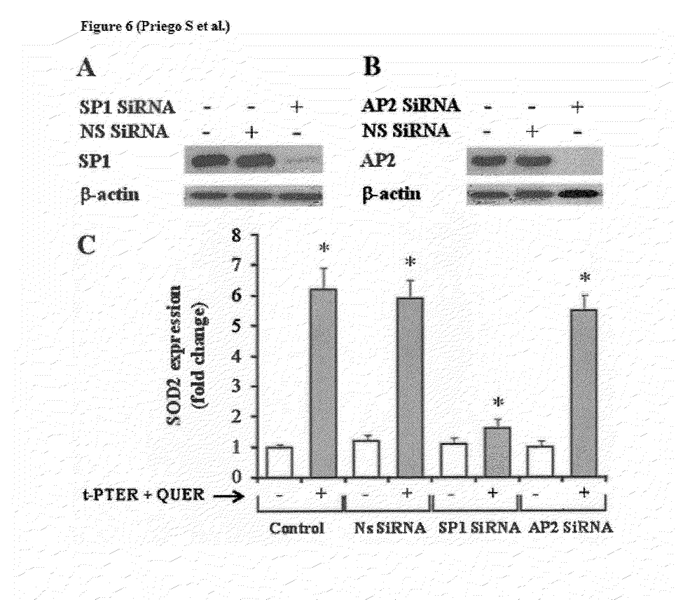

Computer analysis and foot-printing assays revealed a number of putative binding sites for SP1 and AP2 transcription factors in the proximal promoter of human SOD2. These two proteins are main transcriptional regulators of human SOD2 expression but appear to have opposite effects: while the SP1 element positively promotes transcription, the AP2 proteins significantly repress the promoter activity (32). Both, SP1 and AP2 are expressed in HT-29 cells (33). To answer if t-PTER- and QUER-induced increased expression of SOD2 is mediated by these transcriptional regulators, we treated cultured HT-29 cells with t-PTER+QUER and SP1- or AP2-specific siRNA. Western blot analysis shows that SP1 siRNA and AP2 siRNA deplete the intracellular content of their corresponding proteins (FIG. 6. A and B). However, as shown in FIG. 6.C, t-PTER- and QUER-induced up-regulation of SOD2 expression appears mainly dependent on SP1.

### DISCUSSION

t-PTER, a natural analog of t-RESV but 60-100 times stronger as an antifungal agent, shows similar anticarcinogenic properties (34); whereas, QUER may affect tumor cell proliferation, and targets key molecules responsible for tumor cell properties, including e.g. p53 and oncogenic Ras (35-37).

The present inventors have shown that short-time exposure (60 min/day) to bioavailable concentrations of t-PTER and QUER (6), inhibited in vitro growth of HT-29 cells by ˜48% (FIG. 1.A); whereas viability of the remaining cells decreased to ˜71% (>95% in controls) (FIG. 1.B). Loss of cell viability was mainly due to apoptosis. In fact Tinhofer et al. (40) suggested a direct interaction of t-RESV with mitochondria triggering the loss of mitochondrial membrane potential and the opening of the Bcl-2-sensitive pore, and we have shown that t-PTER and QUER induce a NO-dependent inhibition of bcl-2 expression in metastatic cells, thus facilitating the tumor cytotoxicity elicited by the endothelium (6, 41). I.v. administration of t-PTER and QUER (20 mg of each PF/kg×day) also inhibited HT-29 xenograft growth to ˜49% of control values (FIG. 2.B). The association of t-PTER and QUER induced a stronger inhibition of CRC growth than each PF alone (FIG. 2). I.v. administration of 40 mg of t-PTER or QUER/kg×day (n=5 in each case; not shown) induced a CRC growth inhibition which was not significantly different to that shown for the 20 mg/kg×day dose. Thus indicating that the association, and not a higher dose of one, gives better results. In fact, in the B16 melanoma model, t-PTER increases the expression of prodeath BAX and decreases expression of antideath Bcl-2; whereas QUER increases the expression of all prodeath genes analyzed (BAX, BAK, BAD, and BID) and decreases the expression of all antideath genes analyzed (Bcl-2, Bcl-w, and Bcl-xL) (6). Therefore it appears plausible to expect benefits when using the combination.

In vivo treatment with t-PTER and QUER altered expression of molecules involved in regulating cancer cell resistance to drugs and radiations (e.g. the Bcl-2 family of pro-death and anti-death proteins and the antioxidant enzyme system) (FIG. 3). Multidrug and/or radiation resistance are characteristic features of malignant tumors and, in practice, intrinsic (innate) or acquired (adaptive) resistance to therapy critically limits the outcome of cancer patients (42).

The proto-oncogen bcl-2 and its anti-apoptotic homologs are mitochondrial membrane permeabilization inhibitors (43) and participate in development of chemoresistance (21), whereas expression of pro-death genes, e.g. bax or bak, is often reduced in cancer cells (44). As shown in FIG. 3, treatment with t-PTER and QUER significantly increased expression of the pro-apoptotic genes bax, bak, bad, and bid (1.9-2,5-fold); whereas decreased that of the anti-apoptotic bcl-2 (3.3-fold). This is important because e.g. down-regulation of bcl-2 expression can lead to chemosensitization of carcinoma cells [e.g. (45)], and we have shown that antisense oligodeoxynucleotide-induced specific depletion of Bcl-2 facilitates regression of malignant melanoma in mice treated with chemotherapy and ionizing radiations (23).

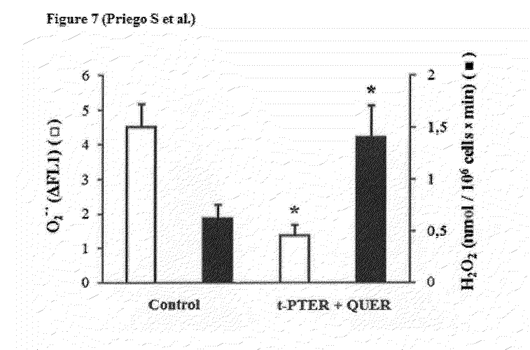

ROS, acting as intracellular second messengers, promote proliferation and maintain the oncogenic phenotype of cancer cells (46). Moreover ROS control the expression of Bcl-2 family proteins by regulating their phosphorylation and ubiquitination (47). A recent report shows that very low concentrations of QUER and rutin (0.1-1 μM) decrease expression of SOD1 and increase that of GPx, thus diminishing ROS (48). However, i.v. administration of t-PTER and QUER (FIG. 3) increased expression of SOD1 (˜1.6-fold), SOD2 (5.7-fold) and CAT, GPx, GR, and TrxR-1 (<2-fold). As shown in FIG. 7, these changes in antioxidant enzymes activities results in H₂O₂ accumulation as compared to controls. A fact that appears in agreement with previous results showing that e.g. tumor suppressive effect of SOD2 overexpression is in part mediated by an antioxidant imbalance resulting in the reduced capacity to metabolize increased levels of intracellular peroxides (26).

Due to a higher production of superoxide anions by the respiratory chain and cytoplasmic NADPH oxidase, the basal concentration of ROS (and particularly H₂O₂) is higher in cancer cells than in their normal counterparts (49). Thus, it is plausible that an increase in SOD2 activity, as here reported (Table 3.A), could cause H₂O₂-induced cytotoxicity and decreased proliferation (49) (see also FIG. 7 showing increased H₂O₂ generation). Huang et al. (50) suggested that malignant cells may be highly dependent on SOD for survival, and proposed SOD activity as a possible target for the selective killing of cancer cells. Numerous in vivo studies show that eventually SODs can be highly expressed in aggressive human tumors, and that high SOD activities have been associated with poor prognosis and resistance to cytotoxic drugs and radiation [see (51) for a review]. However SOD2 overexpression has been correlated in different cancer cell types, including CRC cells, with suppression of neoplastic transformation, decreased proliferation in vitro, and reversion of malignant phenotype (51). Uncoupling of the electrochemical gradient by increased SOD2 activity can give rise to p53 up-regulation and induction of senescence in CRC cells (52); whereas p53-induced suppression of bcl-2 expression can activate the mechanism of cell death (53). Nevertheless HT-29 cells have a mutated p53 (54) and thus, although it may be relevant in other models, a link between SOD2 and p53 in HT-29 cells is unlikely.

As shown in Table 3, combination of t-PTER, QUER, chemotherapy, and radiotherapy eliminated HT-29 cells growing in vivo in most cases (85%, see under Results and the caption to Table 3) leading to long-term survival (>120 days). However, as shown in Table 3, specific overexpression of bcl-2 and/or down-regulation of the SOD2 activity decreased the anti-cancer efficacy of PFs and chemoradiotherapy. Thus proving that key molecules regulate resistance of CRC cells and may determine the efficacy of the therapy.

t-PTER- and QUER-induced down-regulation of bcl-2 expression involves PF-induced inhibition of NF-κB activation, and inhibition of IκBα phosphorylation and degradation (FIG. 4). Indeed natural PFs (including t-RESV, epigallocatechin gallate, or quercetin) are known NF-κB inhibitors (55). Recently we reported that t-PTER and QUER down-regulated inducible NO synthetase, thus causing a NO shortage-dependent decrease in cAMP-response element-binding protein phosphorylation, and a decrease in bcl-2 expression in B16M-F10 cells (41). Active NF-κB participates in the control of transcription of over 150 target genes, including inducible NO synthetase (56), and thus a decrease in endogenous NO generation may be also the link between inhibition of NF-κB activation (FIG. 4) and down-regulation of bcl-2 expression in HT-29 cells (FIG. 3). On the other hand, a wide variety of stimuli can up-regulate SOD2 expression. The cytokine (IL-1, IL-4, IL-6, TNF-α, IFNγ) inducible enhancer regions contain binding sites for NF-κB, C/EBP, and NF-1 transcription factors; whereas protein kinase C stimulating agents, such as the phorbol ester 12-O-tetradecanoylphorbol-13-acetate, induce human SOD2 via a CREB-1/ATF-1 like factor, but not via NF-κB or AP1 (57). Moreover, different microtubule-active anticancer drugs (e.g. paclitaxel or vincristine) may also induce SOD2 expression via activation of protein kinase C and not via NF-κB (58). Here we demonstrate that t-PTER and QUER, which inhibit NF-κB activation (FIG. 4), up-regulate SOD2 expression in human HT-29 cells via a SP1-dependent mechanism (FIG. 6). SP1 positively promotes transcription (32) and its decrease completely prevented the PF-induced increase in SOD2 expression (FIG. 6).

As reviewed by Lamson and Brignall (59), daily i.v. bolus doses of 100 mg QUER/m2 were well tolerated by human patients showing no side-effects or toxicity; whereas i.v. bolus of 1400 mg QUER/m² (approx. 2.5 g in a 70 kg adult) once weekly for three weeks was associated with renal toxicity in two of ten patients. The two patients had a reduction in glomerular flow rate of nearly 20% in the first 24 hours. The reduction resolved within one week, and this effect was not cumulative over subsequent doses in the phase I trial in a population of advanced cancer patients. Transient flushing and pain at the injection site were noted in a dose-dependent manner. Therefore the 1400 mg QUER/m2×week dose was recommended for phase II trials.

Combined administration of PFs and chemoradiotherapy has side effects, as shown by the alterations in hematological and clinical chemistry parameters (Table 2). Nevertheless, such alterations are commonly observed and managed in CRC patients receiving clinical therapies.

Results disclosed herein indicate that using the methods of treatment of the invention it is possible to improve the poor prognosis in a significant number of patients bearing a malignant CRC.

### Example 10

### Evaluation of the Anti-Tumor Activity of Quercetin, Pterostilbene and its Combination in the Presence or Absence of Radiation

A panel of 36 human cell lines were first tested in terms of population doubling time and growth curves to determine the conditions for further assays. The cells were also tested to determine the dose of radiation that produces 50% cell viability (D50).

Table 4 below lists the code, the name and the origin of each of the 36 cell lines and the disease represented by each cell line:

TABLE 4
Cell lines used in the experiments

| Code | Cell line | Origin | Disease |
|---|---|---|---|
| 01 | BT-20 | Breast | Carcinoma |
| 02 | MCF-7 | Breast | Adenocarcinoma |
| 03 | MDA-MB-231 | Breast | Adenocarcinoma |
| 04 | T-47D | Breast | Carcinoma |
| 05 | Colo 201 | Colorectal | Adenocarcinoma |
| 06 | HCT 116 | Colorectal | Carcinoma |
| 07 | HCT-15 | Colorectal | Adenocarcinoma |
| 08 | HT-29 | Colorectal | Adenocarcinoma |
| 09 | HT-1080 | Colorectal | Sarcoma |
| 10 | LN-18 | Brain | Gliblastoma |
| 11 | LN229 | Brain | Gliblastoma |
| 12 | A-172 | Brain | Gliblastoma |
| 13 | U-38 MG | Brain | Gliblastoma/astrocitoma |
| 14 | CHP-126 | Brain | Neuroblastoma |
| 15 | IMR-32 | Brain | Neuroblastoma |
| 16 | SH-SY5Y | Brain | Neuroblastoma |
| 17 | SK-N-AS | Brain | Neuroblastoma |
| 18 | SW-872 | Connective | Fibrosarcoma |
| | | tissue | |
| 19 | HBL-52 | Brain | Meningioma |
| 20 | NCI-H4BO | Lung | Carcinoma |
| 21 | MSTO-211H | Lung | Biphasic Mesothelioma |
| 22 | A549 | Lung | Carcinoma |
| 23 | A375 | Skin | Melanoma |
| 24 | C32 | Skin | Melanoma |
| 25 | SK-MEL-2 | Skin | Melanoma |
| 26 | MEWO | Skin | Melanoma |
| 27 | MML-1 | Skin | Melanoma |
| 28 | KHOS-NP | Bone | Osteosarcoma |
| 29 | MNNG-HOS | Bone | Osteosarcoma |
| 30 | SK-ES-1 | Bone | Osteosarcoma |
| 31 | BxPC-3 | Pancreas | Adenocarcinoma |
| 32 | HPAF-II | Pancreas | Adenocarcinoma |
| 33 | Panc 10 05 | Pancreas | Adenocarcinoma |
| 34 | Panc-1 | Pancreas | Carcinoma Epithiloid |
| 35 | A-204 | Muscle | Rhabdomyosarcoma |
| 36 | A-673 | Muscle | Rhabdomyosarcoma |

To determine IC50 of quercetin, pterostilbene and resveratrol, cells from each cell line were exposed per duplicate to 7 different concentrations of quercetin, pterostilbene, a combination of quercetin and pterostilbene, and resveratrol 24 h post-seeding. Cell viability was determined 120 h after the administration of the compounds using the Cell Counting Kit-8 (Dojindo Molecular Technologies, Inc, Rockville, Md.). In the experiments, 0.1% SDS and 1% DMSO were used as positive and negative controls, respectively. Final concentrations of the compounds used in the experiments are listed below in Table 5:

TABLE 5
Final concentration(s) of the compounds

| Compound(s) | Final concentrations (μM) |
|---|---|
| Pterostilbene | 1, 2, 5, 10, 20, 50, 100 |
| Quercetin | 1, 2, 5, 10, 20, 50, 100 |
| Pterostilbene + Quercetin | 1, 2, 5, 10, 20, 50, 100 |
| (1:1)* | |
| Resveratrol | 1, 2, 5, 10, 20, 50, 100 |

*ratio 1:1 equimolar

To determine the combined effects of the compounds and radiation, cells from each cell line were first administered per duplicate with 7 different concentrations of quercetin, pterostilbene, a combination of quercetin and pterostilbene, and resveratrol 24 h post-seeding. The final concentration of each compound in this set of experiments was also as shown in the above table.

Forty eight (48) hours after the administration of the compounds to the cells, cell cultures were exposed to radiation. Each compound concentration was exposed to 7 different radiation dose levels for every cell line. The radiation dose used in the experiments were 30, 25, 20, 15, 10, 5 and 2.2Gy. Appropriated positive and negative controls were carried out in parallel.

The IC50 of each compound and the combination of quercetin and pterostilbene against each cell line are summarized in Table 6 below:

TABLE 6
IC50 of the compounds

| | IC50 for RI | IC50 for TI1 | IC50 for TI2 | IC50 for TI1 + 2 | D50 for |
|---|---|---|---|---|---|
| Code | (μM) | (μM) | (μM) | (μM) | radiation (Cy) |
| 1 | 66.050 | 96.860 | 9.210 | 23.660 | 1.603 |
| | (R² = 0.9694) | (R² = 0.9878) | (R² = 0.9968) | (R² = 0.9990) | (R² = 0.3930) |
| 2 | 49.880 | 39.430 | 19.730 | 48.470 | >30 |
| | (R² = 0.9985) | (R² = 0.9788) | (R² = 0.9796) | (R² = 0.9985) | |
| 3 | 15.050 | 62.780 | 20.708 | 24.450 | >30 |
| | (R² = 0.9749) | (R² = 0.9422) | (R² = 0.9805) | (R² = 0.9821) | |
| 4 | 25.630 | 35.030 | 10.700 | 21.080 | >30 |
| | (R² = 0.9978) | (R² = 0.9975) | (R² = 0.9952) | (R² = 0.9979) | |
| 5 | 37.400 | 29.790 | 18.060 | 19.300 | >30 |
| | (R² = 2.166) | (R² = 0.9426) | (R² = 0.9713) | (R² = 0.9769) | |
| 6 | 31.380 | 45.210 | 56.930 | 89.900 | 5.465 |
| | (R² = 0.9888) | (R² = 0.9672) | (R² = 0.9828) | (R² = 0.9232) | (R² = 0.9191) |
| 7 | >100 | 49.490 | 12.420 | 32.760 | >30 |
| | | (R² = 0.9508) | (R² = 0.9867) | (R² = 0.9593) | |
| 8 | 27.880 | 28.350 | 18.730 | 31.450 | >30 |
| | (R² = 0.9429) | (R² = 0.9931 | (R² = 0.9795) | (R² = 0.9809) | |
| 9 | 10.230 | 17.780 | 5.539 | 7.900 | 2.324 |
| | (R² = 0.9958) | (R² = 0.9978) | (R² = 0.9974) | (R² = 0.9983) | (R² = 0.9109) |
| 10 | 28.380 | 18.100 | 9.724 | 13.070 | 2.219 |
| | (R² = 0.9983) | (R² = 0.9816) | (R² = 0.9979) | (R² = 0.9982) | (R² = 0.8828) |
| 11 | 11.230 | 14.350 | 8.117 | 15.170 | 2.422 |
| | (R² = 0.9970) | (R² = 0.9469) | (R² = 0.9973) | (R² = 0.9842) | (R² = 0.4259) |
| 12 | 47.650 | 29.700 | 10.020 | 11.750 | >30 |
| | (R² = 0.9831) | (R² = 0.9698) | (R² = 0.9127) | (R² = 0.9821) | |
| 13 | 6.292 | 29.690 | 4.834 | 9.730 | 1.674 |
| | (R² = 0.9521) | (R² = 0.9833) | (R² = 0.9877) | (R² = 0.9752) | (R² = 0.5102) |
| 14 | 26.670 | 1.420 | 19.050 | 4.430 | 1.089 |
| | (R² = 0.9800) | (R² = 0.9984) | (R² = 0.9846) | (R² = 0.9862) | (R² = 0.9824) |
| 15 | 43.010 | 42.780 | 20.170 | 30.520 | 1.714 |
| | (R² = 0.9597) | (R² = 0.9349) | (R² = 0.9927) | (R² = 0.9722) | (R² = 0.9334) |
| 16 | 26.090 | 31.670 | 4.382 | 13.360 | 2.175 |
| | (R² = 0.9945) | (R² = 0.9855) | (R² = 0.9934) | (R² = 0.9938) | (R² = 0.9727) |
| 17 | 68.320 | 29.080 | 19.770 | 27.490 | >30 |
| | (R² = 0.9922) | (R² = 0.9844) | (R² = 0.9887) | (R² = 0.9825) | |
| 18 | 81.120 | 25.750 | 3.514 | 8.263 | 1.8** |
| | (R² = 0.9914) | (R² = 0.9987) | (R² = 0.9915) | (R² = 0.9963) | |
| 19 | 29.060 | 33.160 | 22.770 | 49.370 | >30 |
| | (R² = 0.9934) | (R² = 0.9945) | (R² = 0.9648) | (R² = 0.9864) | |
| 20 | 11.530 | 16.600 | 10.080 | 15.220 | 7.846 |
| | (R² = 0.9459) | (R² = 0.9944) | (R² = 0.9972) | (R² = 0.9937) | (R² = 0.9445) |
| 21 | 6.371 | 12.340 | 19.750 | 18.730 | 2.285 |
| | (R² = 0.9965) | (R² = 0.9930) | (R² = 0.9978) | (R² = 0.9954) | (R² = 0.9458) |
| 22 | 16.330 | 25.790 | 13.430 | 21.400 | 9.179 Gy |
| | (R² = 0.9975) | (R² = 0.9617) | (R² = 0.9851) | (R² = 0.9920) | (R² = 0.7905) |
| 23 | 24.890 | 75.340 | 19.640 | 37.080 | >30 |
| | (R² = 0.9874) | (R² = 0.9737) | (R² = 0.9842) | (R² = 0.9896) | |
| 24 | 18.520 | 30.220 | 22.070 | 32.420 | >30 |
| | (R² = 0.9998) | (R² = 0.9990) | (R² = 0.9931) | (R² = 0.9999) | |
| 25 | 10.700 | 17.290 | 11.610 | 21.710 | 3.808 |
| | (R² = 0.9921) | (R² = 0.9699) | (R² = 0.9947) | (R² = 0.9964) | (R² = 0.5757) |
| 26 | 17.140 | 39.040 | 8.592 | 30.270 | 3.485 |
| | (R² = 0.9619) | (R² = 0.9762) | (R² = 0.9851) | (R² = 0.9840) | (R² = 0.3513) |
| 27 | 10.270 | 16.870 | 10.800 | 19.190 | 3.507 |
| | (R² = 0.9619) | (R² = 0.9762) | (R² = 0.9851) | (R² = 0.9840) | (R² = 0.3513) |
| 28 | 19.960 | 25.620 | 15.710 | 15.990 | 3.084 |
| | (R² = 0.9952) | (R² = 0.9834) | (R² = 0.9866) | (R² = 0.9812) | (R² = 0.7027) |
| 29 | 11.680 | 12.290 | 9.909 | 15.230 | 2.287 |
| | (R² = 0.9993) | (R² = 0.9965) | (R² = 0.9980) | (R² = 0.9861) | (R² = 0.8917) |
| 30 | 27.230 | 42.720 | 8.196 | 9.627 | 2.625 |
| | (R² = 0.9908) | (R² = 0.9596) | (R² = 0.9958) | (R² = 0.9688) | (R² = 0.9234) |
| 31 | 13.59 | 31.06 | 15.88 | 24.16 | 1.449 |
| | (R² = 0.9976) | (R² = 0.9859) | (R² = 0.9891) | (R² = 0.9993) | (R² = 0.7808) |
| 32 | 86.08 | 55.57 | 20.19 | 93.97 | >30 |
| | (R² = 0.6284) | (R² = 0.9748) | (R² = 0.7198) | (R² = 0.9842) | |
| 33 | 24.210 | 46.270 | 22.980 | 48.920 | 13.150 Gy |
| | (R² = 0.9948) | (R² = 0.9931) | (R² = 0.9897) | (R² = 0.9957) | (R² = 0.749)* |
| 34 | 33.040 | 50.150 | 62.460 | 55.490 | >30 |
| | (R² = 0.9883) | (R² = 0.8660) | (R² = 0.9706) | (R² = 0.9343) | |
| 35 | 19.000 | 23.360 | 18.100 | 41.020 | 2.867 |
| | (R² = 0.9949) | (R² = 0.9942) | (R² = 0.9495) | (R² = 0.9919) | (R² = 0.8719) |
| 36 | 6.725 | 5.802 | 4.093 | 4.544 | 7.56** |
| | (R² = 0.9981) | (R² = 0.9979) | (R² = 0.9987) | (R² = 0.9992) | |

*Data calculated with Linear regression.
**Data calculated with semi-log paper.
RI: Resveratrol;
TI1: Pterostilbene;
TI2: Quercetin

### Example 11

### Pterostilbene Inhibits Tumor Cell Growth and Induces Tumor Cell Death

As discussed above, resveratrol has been studied for its anti-diabetic, neuroprotective, anti-adipogenic, cardioprotective and anti-tumoral properties. However its low bioavailability (half-life in circulating blood: ˜14.4 minutes, after i.v. administration of 20 mg/kg to e.g. rabbits) may limit its potential in vivo. Pterostilbene on the other hand, has shown similar or more potent antitumor activities than resveratrol. In addition, pterostilbene has a longer half-life in blood (˜77.9 min). It has been shown that pterostilbene causes cancer cell death in vitro at bioavailable concentrations, and decreases tumor growth in animal models.

The aim of this study was to determine whether pterostilbene causes cytotoxicity in human tumors at concentrations that are reliable under in vivo conditions; and, to identify which death-related molecular mechanisms may be activated by the compound. Human melanoma (A375), breast cancer (MCF7), lung cancer (A549), and colon cancer (HT29) cell lines were used in this set of experiments. Pterostilbene and resveratrol were used in a μM range, between 10 μM (below the IC50 for all cell lines) and 200 μM (an unachievable in vivo concentration). Cell cycle and apoptosis induction were determined by flow cytometry.

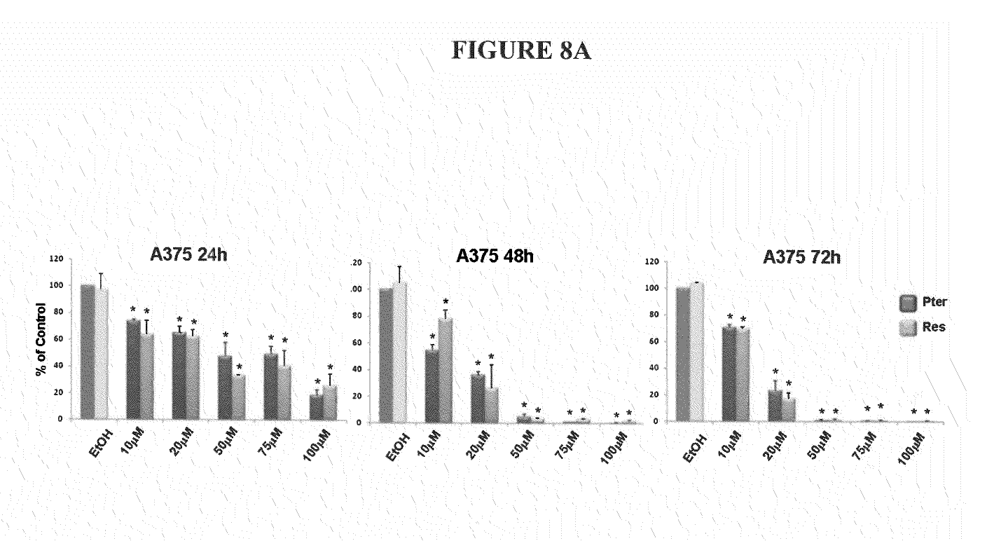

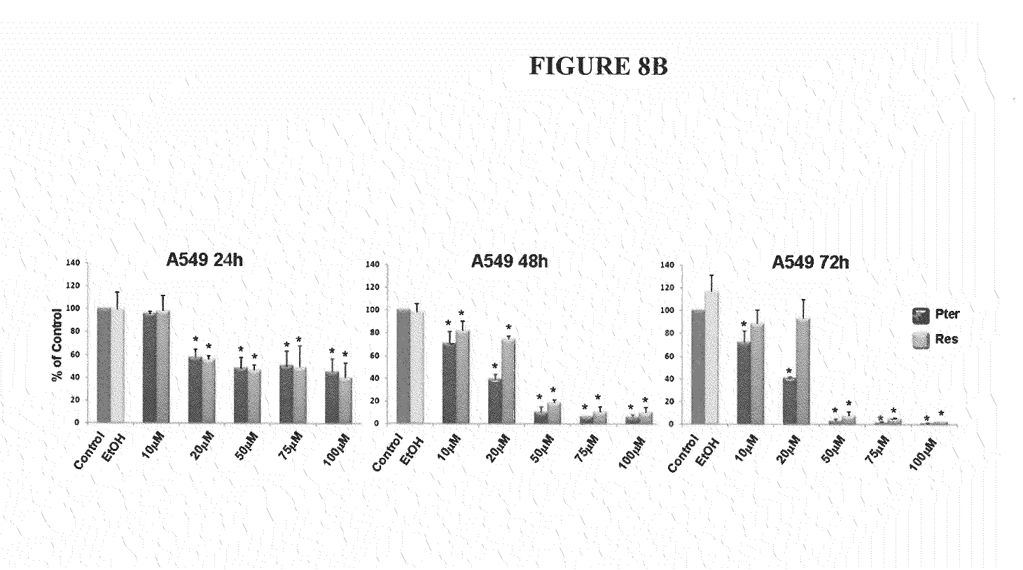

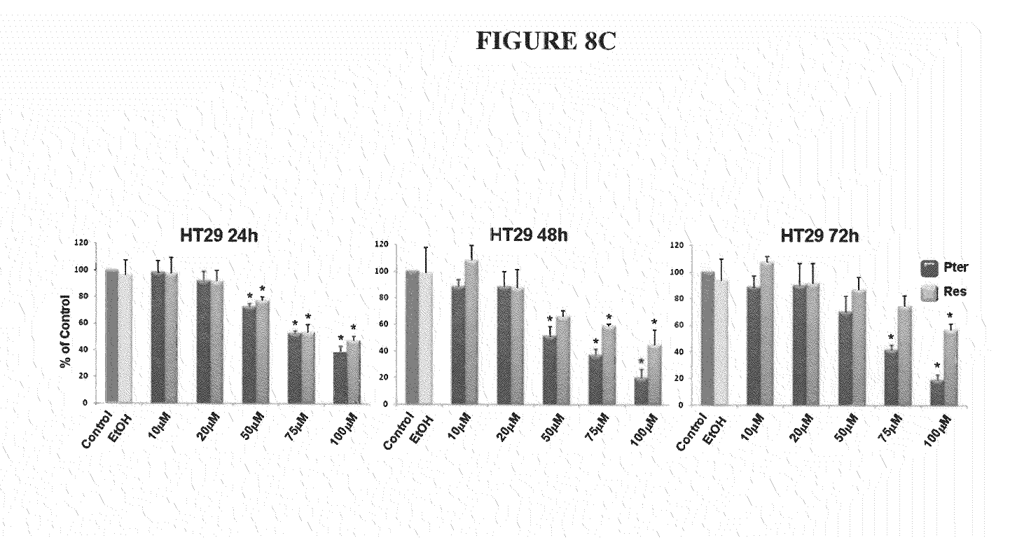

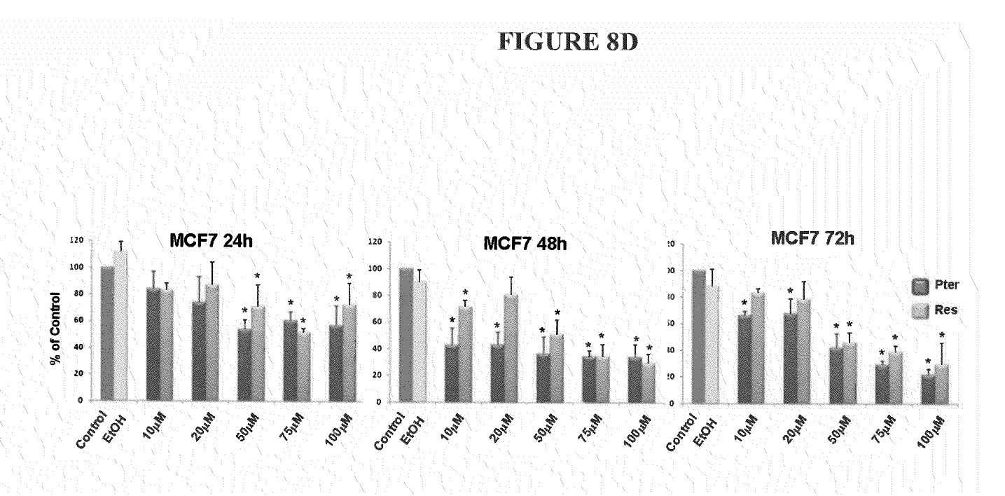

Cells from the cancer lines (0.2×10⁶ cells/well) were seeded in six well-plates and, 24 h later, were treated with pterostilbene or resveratrol (0-100 μM) (ethanol as solvent vehicle was at a conc. of 0.3%). Cell growth was analyzed using the Countess® Automated Cell Counter (Invitrogen). Results were expressed as relative proliferation index±SD (n=4) where control is 100. As can be seen in FIGS. 8A through 8D, the compounds reduced cell number in a concentration- and time-dependent manner.

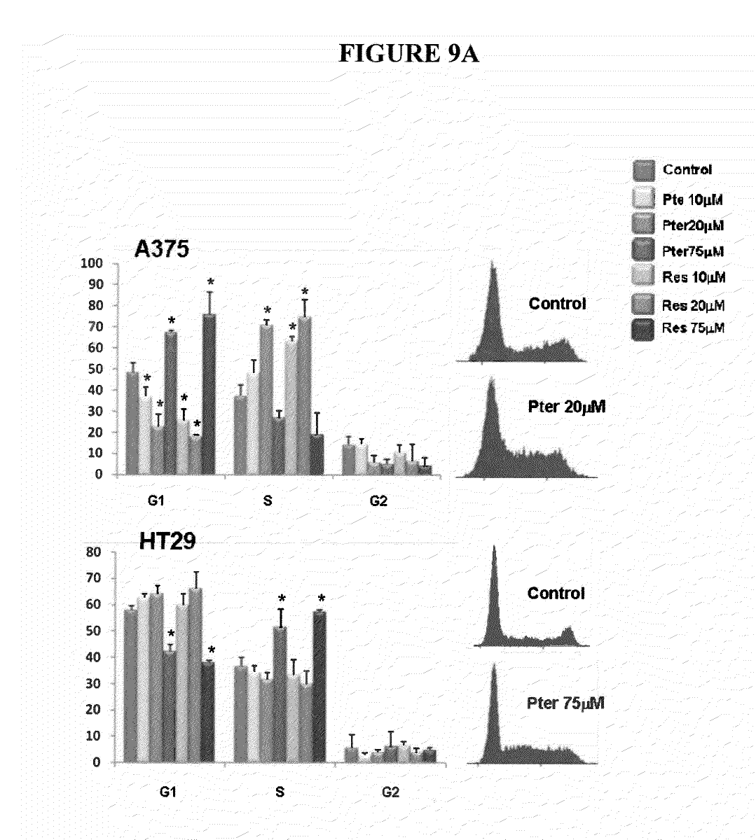

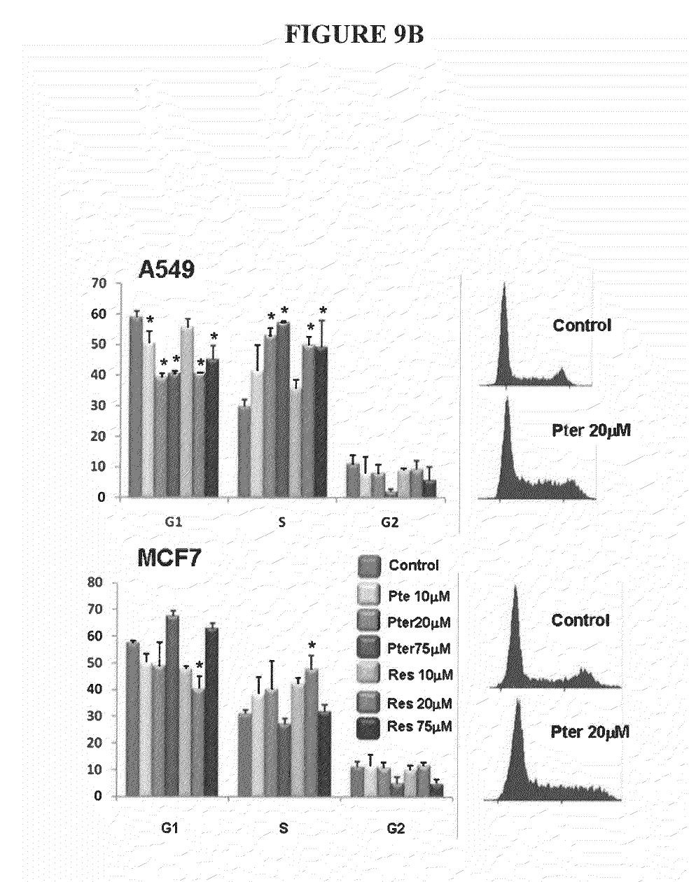

The effects of pterostilbene and resveratrol on cell cycle were tested. The results indicate that pterostilbene or resveratrol induced inhibition of tumor cell division with the cell cycle arrested in S phase. See FIG. 9. Both DNA synthesis and tubulin polymerization were seriously affected. However, no caspase 3 activation was detected within a 24 h-period in the presence of either polyphenol.

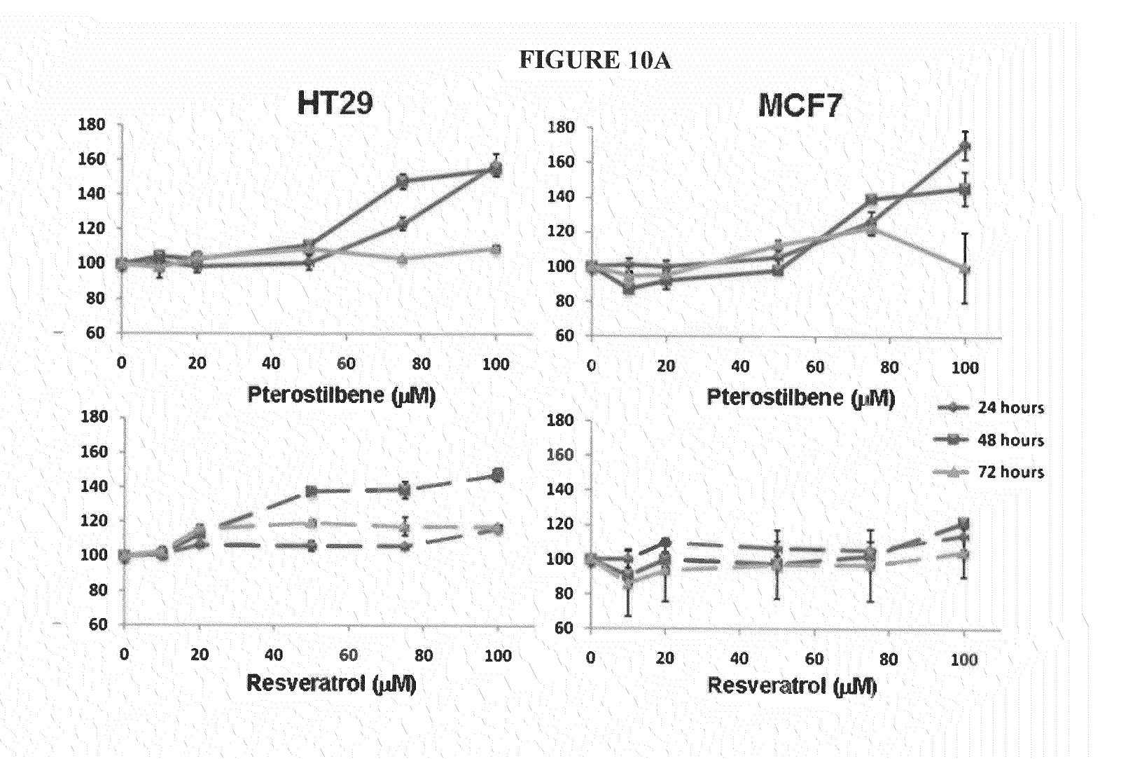

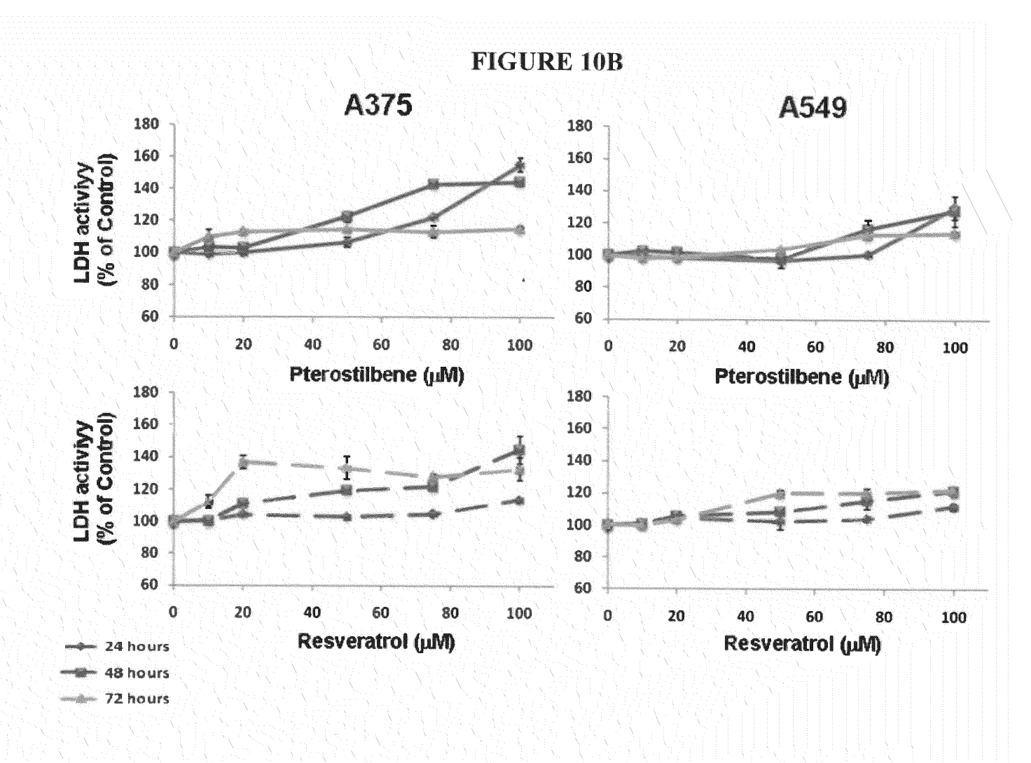

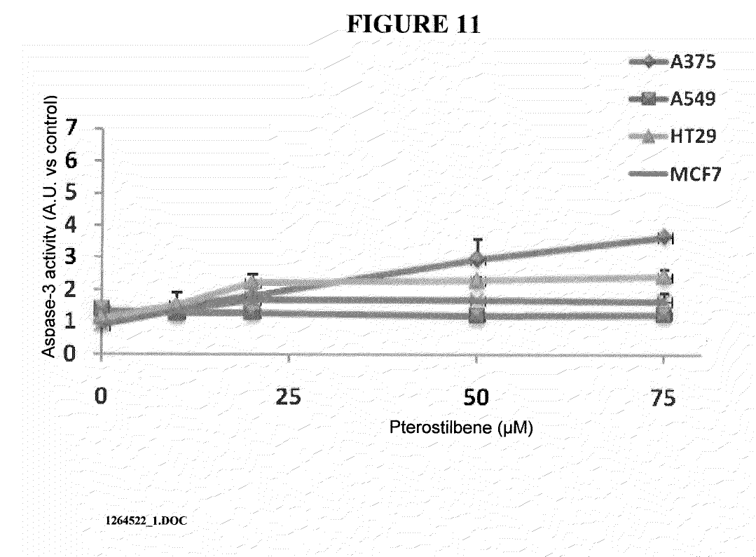

Necrosis was progressively activated with the increase of pterostilbene or resveratrol concentration as evaluated by measuring lactate dehydrogenase (LDH) activity released to the extracellular medium. See FIG. 10. Similarly, apoptosis was progressively activated with the increase of pterostilbene or resveratrol concentration. The tumor cell death induced by pterostilbene appears to be partially independent of caspase activity. See FIG. 11.

In addition, there are different molecular events associating pterostilbene with autophagy activation: a) an increase of LC3-II form, indicating the processing of LC3 protein to its lapidated form; and b) an increase in P62/SQSTM1 bands and GFP-LC3 punctuation, indicating P62 accumulation and translocation of LC3 to autophagic membranes, respectively. An acute loss of tubulin organization, indicating cell cycle arrest, was also detected by immunochemistry. These results demonstrate that pterostilbene activates autophagy in human tumor cells at bioavailable concentrations.

In summary, results from the above experiments demonstrate that pterostilbene or resveratrol inhibits human tumor cell growth in a concentration and time dependent fashion; pterostilbene appears to have a stronger inhibitory effect than that of resveratrol; pterostilbene induces a cell cycle arrest in S phase with both DNA synthesis and tubulin polymerization seriously affected; pterostilbene induces tumor cell death through a mechanism partially independent of caspase activity; and pterostilbene induces autophagy in human tumor cells in a P62/SQSTM1 accumulation dependent pathway.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All patents, patent applications and publications cited herein are hereby incorporated by reference in their entirety. The disclosures of these publications in their entireties are hereby incorporated by reference into this application in order to more fully describe the state of the art as known to one skilled therein as of the date of the invention described and claimed herein.

### REFERENCES

1. Weitz J, Koch M, Debus J, Hohler T, Galle P R, Buchler M W. Colorectal cancer. Lancet 2005; 365:153-65.
2. Meyerhardt J A, Mayer R J. Systemic therapy for colorectal cancer. N Engl J Med 2005; 352:476-87.
3. Thomasset S C, Berry D P, Garcea G, Marczylo T, Steward W P, Gescher A J. Dietary polyphenolic phytochemicals—promising cancer chemopreventive agents in humans? A review of their clinical properties. Int J Cancer 2007; 120:451-8.
4. Jang M, Cai L, Udeani G O, et al. Cancer chemopreventive activity of resveratrol, a natural product derived from grapes. Science 1997; 275:218-20.
5. Asensi M, Medina I, Ortega A, et al. Inhibition of cancer growth by resveratrol is related to its low bioavailability. Free Radic Biol Med 2002; 33:387-98.
6. Ferrer P, Asensi M, Segarra R, et al. Association between pterostilbene and quercetin inhibits metastatic activity of B16 melanoma. Neoplasia 2005; 7:37-47.
7. Ramos S. Effects of dietary flavonoids on apoptotic pathways related to cancer chemoprevention. J Nutr Biochem 2007; 18:427-42.
8. Yang G Y, Liao J, Kim K, Yurkow E J, Yang C S. Inhibition of growth and induction of apoptosis in human cancer cell lines by tea polyphenols. Carcinogenesis 1998; 19:611-6.
9. Yi W, Fischer J, Krewer G, Akoh C C. Phenolic compounds from blueberries can inhibit colon cancer cell proliferation and induce apoptosis. J Agric Food Chem 2005; 53:7320-9.
10. Kim M J, Kim Y J, Park H J, Chung J H, Leem K H, Kim H K. Apoptotic effect of red wine polyphenols on human colon cancer SNU-C4 cells. Food Chem Toxicol 2006; 44:898-902.
11. Obrador E, Navarro J, Mompo J, Asensi M, Pellicer J A, Estrela J M. Glutathione and the rate of cellular proliferation determine tumour cell sensitivity to tumour necrosis factor in vivo. Biochem J 1997; 325 (Pt 1):183-9.
12. Shimizu S, Eguchi Y, Kamiike W, et al. Induction of apoptosis as well as necrosis by hypoxia and predominant prevention of apoptosis by Bcl-2 and Bcl-XL. Cancer Res 1996; 56:2161-6.
13. Gubin A N, Reddy B, Njoroge J M, Miller J L. Long-term, stable expression of green fluorescent protein in mammalian cells. Biochem Biophys Res Commun 1997; 236:347-50.
14. Flohe L, Otting F. Superoxide dismutase assays. Methods Enzymol 1984; 105:93-104.
15. Haag P, Frauscher F, Gradl J, et al. Microbubble-enhanced ultrasound to deliver an antisense oligodeoxynucleotide targeting the human androgen receptor into prostate tumours. J Steroid Biochem Mol Biol 2006; 102:103-13.
16. Bradford M M. A rapid and sensitive method for the quantitation of microgram quantities of protein utilizing the principle of protein-dye binding. Anal Biochem 1976; 72:248-54.
17. Obrador E, Carretero J, Esteve J M, et al. Glutamine potentiates TNF-alpha-induced tumor cytotoxicity. Free Radic Biol Med 2001; 31:642-50.
18. Dignam J D, Lebovitz R M, Roeder R G. Accurate transcription initiation by RNA polymerase II in a soluble extract from isolated mammalian nuclei. Nucleic Acids Res 1983; 11:1475-89.
19. Ortega A L, Carretero J, Obrador E, et al. Tumor cytotoxicity by endothelial cells. Impairment of the mitochondrial system for glutathione uptake in mouse B16 melanoma cells that survive after in vitro interaction with the hepatic sinusoidal endothelium. J Biol Chem 2003; 278:13888-97.
20. Garg A K, Buchholz T A, Aggarwal B B. Chemosensitization and radiosensitization of tumors by plant polyphenols. Antioxid Redox Signal 2005; 7:1630-47.
21. Reed J C. Bcl-2 family proteins: strategies for overcoming chemoresistance in cancer. Adv Pharmacol 1997; 41:501-32.
22. An J, Chervin A S, Nie A, Ducoff H S, Huang Z. Overcoming the radioresistance of prostate cancer cells with a novel Bcl-2 inhibitor. Oncogene 2007; 26:652-61.
23. Mena S, Benlloch M, Ortega A, et al. Bcl-2 and glutathione depletion sensitizes B16 melanoma to combination therapy and eliminates metastatic disease. Clin Cancer Res 2007; 13:2658-66.
24. Grdina D J, Murley J S, Kataoka Y. Radioprotectants: current status and new directions. Oncology 2002; 63 Suppl 2:2-10.
25. Karihtala P, Soini Y. Reactive oxygen species and antioxidant mechanisms in human tissues and their relation to malignancies. Apmis 2007; 115:81-103.
26. Ridnour L A, Oberley T D, Oberley L W. Tumor suppressive effects of MnSOD overexpression may involve imbalance in peroxide generation versus peroxide removal. Antioxid Redox Signal 2004; 6:501-12.
27. Rupnarain C, Dlamini Z, Naicker S, Bhoola K. Colon cancer: genomics and apoptotic events. Biol Chem 2004; 385:449-64.
28. Sarkar F H, Li Y. NF-kappaB: a potential target for cancer chemoprevention and therapy. Front Biosci 2008; 13:2950-9.
29. Aranha M M, Borralho P M, Ravasco P, et al. NF-kappaB and apoptosis in colorectal tumourigenesis. Eur J Clin Invest 2007; 37:416-24.
30. Luo J L, Kamata H, Karin M. IKK/NF-kappaB signaling: balancing life and death—a new approach to cancer therapy. J Clin Invest 2005; 115:2625-32.
31. Nomura M, Ma W, Chen N, Bode A M, Dong Z. Inhibition of 12-O-tetradecanoylphorbol-13-acetate-induced NF-kappaB activation by tea polyphenols, (−)-epigallocatechin gallate and the aflavins. Carcinogenesis 2000; 21:1885-90.
32. Zelko I N, Mariani T J, Folz R J. Superoxide dismutase multigene family: a comparison of the CuZn-SOD (SOD1), Mn-SOD (SOD2), and EC-SOD (SOD3) gene structures, evolution, and expression. Free Radic Biol Med 2002; 33:337-49.
33. Xu Y, Krishnan A, Wan X S, et al. Mutations in the promoter reveal a cause for the reduced expression of the human manganese superoxide dismutase gene in cancer cells. Oncogene 1999; 18:93-102.
34. Rimando A M, Cuendet M, Desmarchelier C, Mehta R G, Pezzuto J M, Duke S O. Cancer chemopreventive and antioxidant activities of pterostilbene, a naturally occurring analogue of resveratrol. J Agric Food Chem 2002; 50:3453-7.
35. Avila M A, Velasco J A, Cansado J, Notario V. Quercetin mediates the down-regulation of mutant p53 in the human breast cancer cell line MDA-MB468. Cancer Res 1994; 54:2424-8.
36. Ranelletti F O, Maggiano N, Serra F G, et al. Quercetin inhibits p21-RAS expression in human colon cancer cell lines and in primary colorectal tumors. Int J Cancer 2000; 85:438-45.
37. Psahoulia F H, Moumtzi S, Roberts M L, Sasazuki T, Shirasawa S, Pintzas A. Quercetin mediates preferential degradation of oncogenic Ras and causes autophagy in Ha-RAS-transformed human colon cells. Carcinogenesis 2007; 28:1021-31.
38. Kuntz S, Wenzel U, Daniel H. Comparative analysis of the effects of flavonoids on proliferation, cytotoxicity, and apoptosis in human colon cancer cell lines. Eur J Nutr 1999; 38:133-42.
39. Lambert J D, Hong J, Yang G Y, Liao J, Yang C S. Inhibition of carcinogenesis by polyphenols: evidence from laboratory investigations. Am J Clin Nutr 2005; 81:284 S-91S.
40. Tinhofer I, Bernhard D, Senfter M, et al. Resveratrol, a tumor-suppressive compound from grapes, induces apoptosis via a novel mitochondrial pathway controlled by Bcl-2. Faseb J 2001; 15:1613-5.
41. Ferrer P, Asensi M, Priego S, et al. Nitric oxide mediates natural polyphenol-induced Bcl-2 down-regulation and activation of cell death in metastatic B16 melanoma. J Biol Chem 2007; 282:2880-90.
42. Pommier Y, Sordet O, Antony S, Hayward R L, Kohn K W. Apoptosis defects and chemotherapy resistance: molecular interaction maps and networks. Oncogene 2004; 23:2934-49.
43. Gross A. BCL-2 proteins: regulators of the mitochondrial apoptotic program. IUBMB Life 2001; 52:231-6.
44. Hickman J A. Apoptosis and tumourigenesis. Curr Opin Genet Dev 2002; 12:67-72.
45. Hussain S, Pluckthun A, Allen T M, Zangemeister-Wittke U. Chemosensitization of carcinoma cells using epithelial cell adhesion molecule-targeted liposomal antisense against bcl-2/bcl-xL. Mol Cancer Ther 2006; 5:3170-80.
46. Storz P. Reactive oxygen species in tumor progression. Front Biosci 2005; 10:1881-96.
47. Li D, Ueta E, Kimura T, Yamamoto T, Osaki T. Reactive oxygen species (ROS) control the expression of Bcl-2 family proteins by regulating their phosphorylation and ubiquitination. Cancer Sci 2004; 95:644-50.
48. Alia M, Mateos R, Ramos S, Lecumberri E, Bravo L, Goya L. Influence of quercetin and rutin on growth and antioxidant defense system of a human hepatoma cell line (HepG2). Eur J Nutr 2006; 45:19-28.
49. Laurent A, Nicco C, Chereau C, et al. Controlling tumor growth by modulating endogenous production of reactive oxygen species. Cancer Res 2005; 65:948-56.
50. Huang P, Feng L, Oldham E A, Keating M J, Plunkett W. Superoxide dismutase as a target for the selective killing of cancer cells. Nature 2000; 407:390-5.
51. Kinnula V L, Crapo J D. Superoxide dismutases in malignant cells and human tumors. Free Radic Biol Med 2004; 36:718-44.
52. Behrend L, Mohr A, Dick T, Zwacka R M. Manganese superoxide dismutase induces p53-dependent senescence in colorectal cancer cells. Mol Cell Biol 2005; 25:7758-69.
53. Kim S S, Chae H S, Bach J H, et al. P53 mediates ceramide-induced apoptosis in SKN-SH cells. Oncogene 2002; 21:2020-8.
54. Zhang W G, Li X W, Ma L P, Wang S W, Yang H Y, Zhang Z Y. Wild-type p53 protein potentiates phototoxicity of 2-BA-2-DMHA in HT29 cells expressing endogenous mutant p53. Cancer Lett 1999; 138:189-95.
55. Nam N H. Naturally occurring NF-kappaB inhibitors. Mini Rev Med Chem 2006; 6:945-51.
56. Pahl H L. Activators and target genes of Rel/NF-kappaB transcription factors. Oncogene 1999; 18:6853-66.
57. Kim H P, Roe J H, Chock P B, Yim M B. Transcriptional activation of the human manganese superoxide dismutase gene mediated by tetradecanoylphorbol acetate. Biol Chem 1999; 274:37455-60.
58. Das K C, Guo X L, White C W. Protein kinase Cdelta-dependent induction of manganese superoxide dismutase gene expression by microtubule-active anticancer drugs. J Biol Chem 1998; 273:34639-45.
59. Lamson D W, Brignall M S. Antioxidants and cancer, part 3: quercetin. Altern Med Rev 2000; 5:196-208.
60. Reagan-Shaw S, Nihal M, Ahmad N. Dose translation from animal to human studies revisited. Faseb J 2008; 22:659-61.
61. Ogata Y, Hara Y, Akagi Y, Ohkita A, Morodomi T, Shirouzu K. Metastatic model of human colon cancer constructed using orthotopic implantation in nude mice. Kurume Med J 1998; 45:121-5.
62. Estrela J M, Obrador E, Navarro J, Lasso De la Vega M C, Pellicer J A. Elimination of Ehrlich tumours by ATP-induced growth inhibition, glutathione depletion and X-rays. Nat Med 1995; 1:84-8.
63. Rimando A M and Suh N., Biological/chemopreventive activity of stilbenes and their effect on colon cancer, Planta Med. 2008 October; 74(13):1635-43. Epub 2008 Oct. 8.
64. Slimestad R., Fossen T, Vågen I M, Onions: a source of unique dietary flavonoids, J Agric Food Chem. 2007 Dec. 12; 55(25):10067-80. Epub 2007 Nov. 13.
65. Paciotti G F, Myer L, Weinreich D, Goia D, Pavel N, McLaughlin R E, Tamarkin L., L Colloidal gold: a novel nanoparticle vector for tumor directed drug delivery, Drug Deliv. 2004 May-June; 11(3):169-83.

1. A method for treating cancer in a subject in need thereof comprising,
co-administering to said subject a therapeutically effective amount of pterostilbene and quercetin,
wherein said cancer is characterized by overexpression or constitutive activation of NF-κB or Bcl-2.

2. The method of claim 1, wherein said treatment has a cytostatic effect on said cancer.

3. The method of claim 1, wherein said treatment has a cytotoxic effect on said cancer.

4. The method of claim 3, wherein said treatment further comprises administering an additional therapeutic agent.

5. The method of claim 4, wherein said additional therapeutic agent is a polyphenol.

6. The method of claim 5, wherein said polyphenol is resveratrol.

7. The method of claim 5, wherein said additional polyphenol is selected from the group consisting of: TMS, 3,4′,4-DH-5-MS, 3,5-DH-4′MS, catechin, caffeic, hydroxytyrosol, rutin, and quercitrin.

8. The method of claim 1, wherein said cancer is selected from the group consisting of skin cancer, colon cancer, advanced colorectal cancer, breast cancer, prostate cancer, lung cancer, uveal melanoma, brain cancer, lung cancer, bone cancer, pancreas cancer, fibrosarcoma and rhabdomyosarcoma.

9. The method of claim 1, wherein said cancer is colon cancer or advanced colorectal cancer.

10. The method of claim 1 further comprising treating said subject with chemotherapy or a radiation therapy.

11. The method of claim 1 further comprising treating said subject with chemotherapy and a radiation therapy.

12. The method of claim 11, wherein said treatment causes regression of said cancer.

13. The method of claim 10, wherein said treatment has no systemic toxicity in said subject.

14. The method of claim 10, wherein said chemotherapy uses an agent selected from the group consisting of: oxaliplatin, fluorouracil, leucovorin, 5-fluorouracil, leucovorin, and irinotecan.

15. The method of claim 10, wherein said chemotherapy is an irinotecan-based chemotherapy or an oxaliplatin-based chemotherapy.

16. The method of claim 15, wherein said chemotherapy comprises administering to said subject a combination of oxaliplatin, fluorouracil and leucovorin, or a combination of 5-fluorouracil, leucovorin, and irinotecan.

17. The method of claim 1, wherein said pterostilbene and quercetin are administered orally.

18. The method of claim 1, wherein said pterostilbene and quercetin are administered intravenously.

19. A method for treating colorectal cancer in a subject in need thereof comprising,
co-administering to said subject a therapeutically effective amount of pterostilbene and quercetin,
wherein said treatment inhibits cancer cell growth or kills cancer cells.

20. The method of claim 19 further comprising treating said subject with a radiation therapy or a chemotherapy.

21. The method of claim 19, further comprising treating said subject with a radiation therapy and a chemotherapy.

22. The method of claim 20, wherein said chemotherapy is an irinotecan-based chemotherapy or an oxaliplatin-based chemotherapy.

23. The method of claim 22, wherein said chemotherapy comprises administering to said subject a combination of oxaliplatin, fluorouracil and leucovorin, or a combination of 5-fluorouracil, leucovorin, and irinotecan.

24. The method of claim 20, wherein said treatment causes regression of said cancer in said subject.

25. A method for treating colorectal cancer in a subject comprising,
co-administering to said subject a therapeutically effective amount of pterostilbene and quercetin in combination with a chemotherapy or a radiation therapy.

26. A method for treating colorectal cancer in a subject in need thereof comprising,
co-administering to said subject a therapeutically effective amount of pterostilbene and quercetin in combination with a chemotherapy and a radiation therapy
wherein said treatment causes regression of said cancer.

27. The method of claim 25, wherein said chemotherapy comprises administering to said subject a combination of oxaliplatin, fluorouracil and leucovorin, or a combination of 5-fluorouracil, leucovorin, and irinotecan.

28. The method of claim 25, wherein said pterostilbene and quercetin are administered intravenously.

29. The method of claim 25, wherein said treatment has no systemic toxicity to said subject.

30. The method of claim 1, 19, 25 or 26, wherein said subject is a human.

31. The method of claim 1, 19, 25 or 26, wherein said treatment delivers a dose of quercetin of 800 mg/m² and a dose of pterostilbene of 800 mg/m² to said subject.

32. The method of claim 1, wherein said pterostilbene and quercetin are administered concurrently.

33. The method of claim 1, wherein said pterostilbene and quercetin are administered sequentially.
